SEQUENCE LISTING (10 sequences)

SEQ ID NO: 1 - protein, 10 aa, Homo sapiens
ELAGIGILTV

SEQ ID NO: 2 - protein, 15 aa, Homo sapiens
KLPTLAKFSPYLGQM

SEQ ID NO: 3 - protein, 15 aa, Homo sapiens
TSQFLSLQCLQALYV

SEQ ID NO: 4 - protein, 15 aa, Homo sapiens
LSLQCLQALYVDSLF

SEQ ID NO: 5 - protein, 15 aa, Homo sapiens
HLIGLSNLTHVLYPV

SEQ ID NO: 6 - protein, 15 aa, Homo sapiens
LSNLTHVLYPVPLES

SEQ ID NO: 7 - protein, 9 aa, Homo sapiens
TLAKFSPYL

SEQ ID NO: 8 - protein, 10 aa, Homo sapiens
SLQCLQALYV

SEQ ID NO: 9 - protein, 10 aa, Homo sapiens
SNLTHVLYPV

SEQ ID NO: 10 - protein, 9 aa, Homo sapiens
NLTHVLYPV

(19) United States
(12) Patent Application Publication
(10) Pub. No.: US 2022/0033766 A1
(43) Pub. Date: Feb. 3, 2022

(54) ANTIGEN-SPECIFIC T LYMPHOCYTES AND METHODS OF MAKING AND USING THE SAME

(71) Applicant: Torque Therapeutics, Inc., Cambridge, MA (US)

(72) Inventors: James Andrew Rakestraw, Somerville, MA (US); Shawn Carey, Maynard, MA (US)

(21) Appl. No.: 17/274,943

(22) PCT Filed: Sep. 10, 2019
(86) PCT No.: PCT/US19/50492

§ 371 Date: Mar. 10, 2021

Related U.S. Application Data
(60) Provisional application No. 62/729,220, filed on Sep. 10, 2018.
(60) Provisional application No. 62/884,527, filed on Aug. 8, 2019.

(51) Int. Cl. C12N 5/00; C12N 5/0784; C12N 5/0783; A61K 35/17

(52) U.S. Cl. CPC C12N 5/0006; C12N 5/0639; C12N 5/0638; C12N 2502/1114; C12N 2501/10; C12N 2501/20; A61K 35/17

(57) ABSTRACT

Methods and compositions disclosed herein relate to cancer immunotherapy, in particular preparation and use of antigen-specific T lymphocytes for immune cell therapies. Methods and compositions disclosed herein relate to the production of antigen-presenting cells and their use cell therapy and vaccines and, in particular, the preparation and use of antigen-specific T lymphocytes for cancer immunotherapies.

### CROSS REFERENCE TO RELATED APPLICATIONS

This application claims priority to and the benefit of U.S. Provisional Patent Application Nos. 62/729,220 filed Sep. 10, 2018 and 62/884,527 filed Aug. 8, 2019, each of which is incorporated herein by reference in its entirety.

### FIELD

Methods and compositions disclosed herein relate to the production of antigen-presenting cells and their use cell therapy and vaccines and, in particular, the preparation and use of antigen-specific T lymphocytes for cancer immunotherapies.

### BACKGROUND

Immune cell therapies, e.g., adoptive cell therapy (ACT), typically include the steps of collecting immune cells from a subject, expanding the cells, and reintroducing the cells into the same subject or a different subject. For example, ACT of donor-derived, ex-vivo expanded human antigen-specific multi-targeted T cells (MTCs) has emerged as a promising approach to treat cancer. Other ACT approaches include cultured tumor infiltrating lymphocytes (TILs), isolated and expanded T cell clones, and genetically engineered lymphocytes (e.g., T cells) that express conventional T cell receptors or, in the case of CAR-T therapy, chimeric antigen receptors. Genetically engineered lymphocytes are designed to eliminate cancer cells expressing specific antigen(s) and are expanded ex vivo before being delivered to a patient. ACT can provide tumor-specific lymphocytes (e.g., T cells) that lead to a reduction in tumor cells in a patient.

Conventional methods for MTC preparation, however, suffer many limitations and drawbacks, such as low activation rates or low reactivity; or suffer from suboptimal process limitations such as multiple fresh blood draws, complexity of procedures, lack of standardization, multi-day dendritic cell generation cycle, etc. As such, a need exists for improved methods and compositions for MTC preparation.

### SUMMARY

The present disclosure described herein provides, in some aspects, improved methods and compositions related to the in vitro generation antigen-presenting cells (APCs) and MTCs trained by such APCs for use in cell therapeutics, vaccines, and immunotherapeutics. Such methods and compositions may be used in the treatment or prevention of, for example, cancer, auto-immune diseases or disorders, or viral infections. According to the invention, APCs are generated by culturing monocytes under conditions suitable for differentiation into immature dendritic cells (iDCs) and maturation of the (iDCs) into fully mature dendritic cells (mDCs). The monocytes and/or iDCs are contacted with one or more antigens, antigenic proteins, and/or libraries of antigen peptides under conditions suitable for internalization of such antigens, antigenic proteins and/or peptides by the monocytes and/or iDCs where at least a portion of the antigens, proteins and/or peptides will be subjected to proteolytic processing and, ultimately, presentation on the cell surface of the mDCs to produce APCs. Certain embodiments provide compositions comprising novel populations of APCs characterized by the presentation of the proteolytically processed antigens, proteins and/or peptides, as well as dendritic cell therapeutics and vaccines comprising such APCs. Also provided herein are compositions comprising novel populations of T cells primed or trained by such APCs, as well as T cell therapeutics comprising such T cells.

In one aspect, provided herein is a method for preparing antigen-presenting cells (APCs), the method comprising:

(a) contacting a plurality of monocytes and/or immature dendritic cells with a library of peptides in a medium under suitable conditions for the monocytes and/or immature dendritic cells to internalize one or more of the peptides, wherein the library of peptides comprises one or more full-length antigen and/or peptide fragments of an antigen, and wherein the peptides are suitable for proteolytic processing by the monocytes and/or immature dendritic cells and subsequent loading onto at least one dendritic cell surface protein complex selected from a class I major histocompatibility complex (MHC I) and/or a class II major histocompatibility complex (MHC II); and

(b) culturing the monocytes and/or immature dendritic cells in the presence of one or more cytokines and/or growth factors under suitable conditions to induce differentiation of the monocytes and/or maturation of the immature dendritic cells into mature antigen-presenting dendritic cells, thereby to prepare APCs.

In some embodiments, each peptide in the library of peptides comprises 5 or more, 8 or more, 10 or more, 15 or more, 20 or more, 5, 8, 9, 10, 11, 12, 13, 14, 15, 16, 17, 18, 19, 20, 10-15, 5-10, 5-15, 5-20, 8-10, 8-15, 8-20, 10-20, 15-20, 10-100, 10-150, 10-200, or longer than 200 amino acids. In certain embodiments, one or more full-length antigens can be included in the library. In some embodiments, the library of peptides comprises peptide or protein fragments of more than one antigen. In some embodiments, the library of peptides comprises fragments of 1, 2, 3, 4, 5, 6, 7, 8, 9, 10, 2-5, 2-10, 3-10, 4-10, 5-10, at least 1, at least 2, at least 3, at least 4, at least 5, or at least 6 antigens.

In various embodiments, the method can be used to prepare APCs using full-length protein of one or more antigen or fragments larger that small peptides of one or more antigen. In this regard, the method is not limited to using a library or peptides.

In some embodiments, the step of contacting the monocytes and/or immature dendritic cells comprises contacting the monocytes and/or immature dendritic cells with 2 or more libraries of peptides, wherein each library of peptides comprises fragments of a different antigen. In some embodiments, step (a) comprises contacting the monocytes and/or immature dendritic cells with 1, 2, 3, 4, 5, 6, 7, 8, 9, 10, 2-5, 2-10, 3-10, 4-10, 5-10, at least 1, at least 2, at least 3, at least 4, at least 5, or at least 6 libraries of peptides. In some embodiments, the libraries of peptides include a library of peptides comprising fragments of a tumor-associated antigen, a library of peptides comprising fragments of a viral tumor-associated antigen, or both.

In some embodiments, the antigen is a tumor-associated antigen. In some embodiments, the antigen is a viral tumor-associated antigen. In some embodiments, the library of peptides comprises antigens that are tumor-associated antigens, viral tumor-associated antigens, or both.

In some embodiments, the method further includes:

(c) contacting the APCs with a library of peptides in a medium under suitable conditions for the APCs to load the peptides onto at least one cell surface protein complex selected from an MHC I and/or an MHC II, wherein the library of peptides comprises peptide fragments of an antigen, and

(d) optionally repeating step (c) one or more times.

In some embodiments, the library of peptides comprises a plurality of peptides having a length of 5 or more, 8 or more, 10 or more, 15 or more, 20 or more, 5, 8, 9, 10, 11, 12, 13, 14, 15, 16, 17, 18, 19, 20, 23, 25, 10-15, 5-10, 5-15, 5-20, 5-25, 8-10, 8-15, 8-25, 10-25, 15-25, 10-100, 10-150, 10-200, or longer than 200 amino acids. In certain embodiments, one or more full-length antigens can be included in the library. In some embodiments, the library of peptides comprises peptide fragments of more than one antigen. In some embodiments, the library of peptides comprises fragments of 1, 2, 3, 4, 5, 6, 7, 8, 9, 10, 2-5, 2-10, 3-10, 4-10, 5-10, at least 1, at least 2, at least 3, at least 4, at least 5, or at least 6 antigens.

In some embodiments, the step of contacting the ACPs with a library of peptides comprises contacting the APCs with 2 or more libraries of peptides, wherein each library of peptides comprises a different antigen, or fragments thereof. In some embodiments, the step comprises contacting the APCs with 1, 2, 3, 4, 5, 6, 7, 8, 9, 10, 2-5, 2-10, 3-10, 4-10, 5-10, at least 1, at least 2, at least 3, at least 4, at least 5, or at least 6 libraries of peptides. In some embodiments, the libraries of peptides include a library of peptides comprising a tumor-associated antigen or fragments thereof, a library of peptides comprising a viral tumor-associated antigen or fragments thereof, or both.

In some embodiments, at least one of the library or libraries of peptides of step (c) described above is the same as at least one of the library or libraries of peptides of step (a) described above. In some embodiments, at least one of the library or libraries of peptides of step (c) is different than at least one of the library or libraries of peptides of step (a). In some embodiments, at least one of the libraries of peptides comprises peptide fragments of a tumor-associated antigen, a viral tumor-associated antigen, or both.

In some embodiments, step (a) described above comprises contacting the monocytes and/or immature dendritic cells with a library of peptides in a medium at a first concentration, and wherein step (c) described above comprises contacting the APCs with a library of peptides in a medium at a second concentration, wherein the first concentration is the same as, more than, or less than the second concentration.

In some embodiments, the peptides comprise one or more tumor-associated antigens, or fragments thereof, selected from the group consisting of PRAME, SSX2, NY-ESO-1, Survivin, WT-1 and MART. In some embodiments, the peptides can include the following tumor-associated antigens, or fragments thereof. PRAME, NY ESO-1, WT-1, SSX-2, and Survivin. In certain embodiments, the tumor-associated antigens can additionally include viral tumor antigens for, e.g., HPV⁺ head & neck cancer and/or cervical cancer. In some embodiments, the peptides can be from mutated proteins, such as neoantigen peptides.

In some embodiments, the method can further include contacting the APCs with a plurality of T cells under conditions suitable for antigen-priming and/or antigen-specific activation of the T cells, thereby to produce a population of T cells comprising primed and/or activated T cells specific for the antigen presented by the APCs. In some embodiments, the APCs are contacted with the plurality of T cells in the presence of at least one of the libraries of peptides of step (a) and/or step (c). In some embodiments, the method further includes culturing the population of T cells in the presence of one or more cytokines and/or growth factors under conditions suitable to induce proliferation of the T cells. In some embodiments, the population of T cells is cultured in the presence of at least one of the libraries of peptides of step (a) and/or step (c). In some embodiments, the method further includes culturing the population of T cells in the presence of the APCs suitable for antigen-priming and/or antigen-specific activation of the T cells. In some embodiments, the step of contacting the APCs with the primed and/or activated T cells is repeated one or more times. In some embodiments, the primed and/or activated T cells are co-cultured with the APCs.

Another aspect relates to a method for preparing antigen-specific T Cells (APCs), the method comprising:

(a) contacting a plurality of monocytes and/or immature dendritic cells with a library of peptides in a medium under suitable conditions for the monocytes and/or immature dendritic cells to internalize one or more of the peptides, wherein the library of peptides comprises peptide fragments of an antigen, and wherein the peptides are suitable for proteolytic processing and loading by the monocytes and/or immature dendritic cells onto at least one cell surface protein complex selected from a class I major histocompatibility complex (MHC I) and/or a class II major histocompatibility complex (MHC II);

(b) culturing the monocytes and/or immature dendritic cells in the presence of one or more cytokines and/or growth factors under suitable conditions to induce differentiation of the monocytes and/or maturation of the immature dendritic cells into mature antigen-presenting dendritic cells, thereby to prepare APCs;

(c) contacting a plurality of T cells with the APCs under conditions suitable for antigen-priming and/or antigen-specific activation of the T cells, thereby to prepare a population of T cells comprising primed and/or activated T cells specific for the antigen presented by the APCs;

(d) contacting the population of T cells prepared in step (c) with APCs under conditions stimulate the primed and/or activated T cells; and

(e) optionally, repeat step (d) one or more times.

In some embodiments, the contacting step (c) comprises culturing the plurality of T cells with the APCs in the presence of a library of peptides. In some embodiments, the library of peptides of the contacting step (c) is the same as the library of peptides of the contacting step (a). In some embodiments, the library of peptides of the contacting step (c) is different than the library of peptides of step (a). In some embodiments, step (d) comprises culturing the population of T cells with the APCs in the presence of a library of peptides. In some embodiments, the library of peptides of the contacting step (d) is the same as the library of peptides of the contacting step (a). In some embodiments, the library of peptides of the contacting step (d) is different than the library of peptides used in the contacting step (a).

A further aspect relates to a composition comprising: a population of APCs prepared according to the method disclosed herein, wherein the APCs comprise a plurality of MHC I and/or MHC II complexes loaded with processed peptides, wherein preferably the processed peptides have been shortened in vivo by, e.g., monocytes and/or iDCs before presentation on the MHC I and/or MHC II complexes. In some embodiments, the processed peptides are shorter in length than the peptides in the library of peptides. In some embodiments, the processed peptides are less than 8, less than 10, less than 12, less than 15, between 5 and 15, between 8 and 10, between 8 and 12, between 8 and 14, or between 8 and 15 amino acids in length. In some embodiments, the composition can further include a plurality of mDCs loaded with peptides from the library of peptides (using, e.g., conventional loading).

Another aspect relates to a composition comprising: a first population of APCs prepared according to the method disclosed herein, wherein the first population of APCs comprise a plurality of MHC I and/or MHC II complexes loaded with processed peptides, and a second population of APCs comprising a plurality of MHC I and/or MHC II complexes loaded with peptides from the library of peptides.

A further aspect relates to a composition comprising: a first population of APCs presenting a first plurality of peptides having at least 2 different lengths, wherein preferably the first plurality of peptides has been shortened in vivo by, e.g., iDCs before presentation on the first population of APCs; and a second population of APCs presenting a second plurality of peptides having a uniform length. In some embodiments, the first population of APCs and the second population of APCs are present at a ratio of about 1:1.

Also provided herein is a composition comprising an expanded population of monocyte-derived APCs, wherein each APC comprises at least one MHC complex loaded with a peptide from the same antigen, and wherein as a population, the APCs comprise a plurality of MHC complexes loaded with a plurality of peptides from said antigen, the plurality of peptides being of different lengths. In some embodiments, each of the plurality of peptides has a length of 5 or more, 6 or more, 7 or more, 8 or more, 10 or more, 15 or more, 20 or more, between 5 and 10, between 5 and 15, between 5 and 20, between 6 and 10, or between 6 and 20 amino acids. In certain embodiments, as a population, the APCs further comprise a second plurality of MHC complexes loaded with a plurality of peptides from a second antigen, the second plurality of peptides having the same length. In some embodiments, the first antigen and the second antigen are the same antigen.

A further aspect relates to a composition comprising: a population of primed and/or activated T cells prepared according to the method disclosed herein, wherein the primed and/or activated T-cells comprise a plurality of MHC I and/or MHC II complexes loaded with processed peptides.

Another aspect relates to a composition comprising: a population of primed and/or activated T cells prepared according to the method disclosed herein, wherein the primed and/or activated T-cells comprise a plurality of MHC I and/or MHC II complexes loaded with processed peptides, and a plurality of MHC I and/or MHC II complexes loaded with peptides from the library of peptides. In some embodiments, the population of primed and/or activated T cells comprise CD4+ T cells, CD8+ T cells, and/or cytotoxic T lymphocytes (CTLs).

Also provided herein is a pharmaceutical composition comprising any one of the compositions disclosed herein, and a pharmaceutically acceptable carrier or excipient.

A further aspect relates to a method of treating cancer, comprising administering the pharmaceutical composition disclosed herein to a patient in need thereof.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is an overview of the platform technology for producing highly potent multi-target T cells (MTC). Briefly, T cells and monocytes are isolated from patient apheresis. The monocytes are converted to mature dendritic cells (mDCs) which display tumor-associated antigen (TAA) peptides. These mDCs are used to expand (e.g. prime) the population of TAA-reactive T cells. The T cells can then be Deep Primed™ by loading with immunomodulating cytokines before re-infusion into the patient.

FIG. 2 shows an embodiment in which a combination DC pool is created by combining conventional mature DCs loaded directly with 15mer peptides and preloaded DCs that present 6-15mer peptides.

FIG. 3A is a valuation of antigen processing and presentation using MART1 tool peptides and MART1-specific single chain TCR reagent.

FIG. 3B shows an example of HLA-A*02:01 presentation of MART1 10mer peptide by dendritic cells. Mature dendritic cells were loaded with MART1 peptide by incubating cells with MART1 10mer, 15mer, or 23mer on day 3 following maturation (Conventional). Alternatively, the MART1 peptides were added to monocytes on day 0 (D0 preloading) or immature dendritic cells on day 1 (D1 preloading) of the dendritic cell differentiation and maturation process. Surface presentation of HLA-A*02:01/MART1 10mer is detected by fluorophore-labeled multimeric single chain TCR reagent.

FIG. 3C shows the impact of preloading dose on presentation of MART1 10mer peptide-HLA-A*02:01 by dendritic cells. Dendritic cells were loaded by adding the MART1 15mer at the indicated concentration on day 0 or day 1 of the dendritic cell differentiation and maturation process. Surface presentation of HLA-A*02:01/MART1 10mer is detected by fluorophore-labeled multimeric single chain TCR reagent. Representative data from two healthy HLA-A*02:01 donors.

FIG. 4A is a schematic of MTC training through dendritic cell preloading. Monocytes or immature dendritic cells were preloaded with 15mer peptides during differentiation and maturation to mature dendritic cells and were subsequently used to train MART1 10mer-specific T cells.

FIG. 4B shows the detection of MART1 10mer-specific T cells by peptide-MHC tetramer.

FIG. 4C shows mature dendritic cells preloaded with MART1 15mer stimulate enrichment of MART1 10mer-specific T cells during a 14-day, 2-stimulation T cell training co-culture. Representative data from two healthy HLA-A*02:01 donors.

FIG. 4D shows dendritic cells preloaded with MART1 15mer stimulate preferential expansion and enrichment of MART1 10mer-specific T cells over nonspecific bystander T cells. Representative data from two healthy HLA-A*02:01 donors.

FIG. 5 shows complementary tumor-associated antigens and viral tumor-associated antigens can drive enrichment of cognate MTCs and anti-tumor immune responses by natural T cells.

FIG. 6 is a scheme of DC generation and T cell priming to compare methods of antigen loading.

FIG. 7 shows MTC generated using DC loaded with TAA peptides by conventional, preloading, or combinations (1:1 mixture of conventional and preloading) are assessed for reactivity to TAA using a co-culture T cell activation assay. Reactivity is reported for MTC products from 3 healthy donors harvested on day 14 (2 DC stimulation process) or day 21 (3 DC stimulation process). Dashed lines indicate a standard 21-day conventionally loaded process results.

FIG. 8 shows TAA reactivity in CD3, CD4, and CD8 compartments for MTC generated as in FIG. 7.

FIG. 9 shows reactivity of MTC trained using conventional or combination conventional/preloaded DCs measured by IFN-γ production by activated T cells upon stimulation with TAA/MART1 15mer peptide pool or the MART1 10mer.

FIG. 10A shows an embodiment in which a pool of 5 TAA libraries containing 356 unique 15mer peptides is added to monocytes which are matured to immature DCs and mature DC. The resulting peptide-loaded mature DCs are co-cultured with autologous T cells to enrich for TAA-specific MTC. The harvested MTC product is assessed for reactivity to selected PRAME-derived 9mer and 10mer peptides via staining with fluorophore-conjugated peptide-loaded MHC I tetramer.

FIG. 10B shows the interrogation of MTCs trained against TAA using a combination process for binding to PRAME-derived 9mer and 10mer peptide via peptide-loaded MHC tetramers (MTC binding to a pool of the four PRAME tetramers is shown at left). The population of tetramer-binding, CD8 cells is highlighted. CD8 reactivity to the individual peptides is shown at right.

FIG. 11 shows the T cell activation and IFN-γ secretion in response to stimulation with autologous DCs and partially HLA-matched cancer cells. Activation data are presented as the percent of T cells showing CD25 expressing above T cell only culture, and error bars indicate the range of response to two independent pools peptide-loaded DCs. Dashed lines indicate response to non-loaded DC. IFN-γ ELISA ULOQ=1×1e4 pg/mL.

### DETAILED DESCRIPTION

The present disclosure provides herein, in some embodiments, methods for preparing antigen-presenting cells (APCs) and uses of such APCs in, for example, the preparation of cell therapeutics and vaccines. Also provided, in some embodiments are compositions comprising APCs and/or T cells prepared according to the methods of the present disclosure, as well as certain pharmaceutical preparations, cell therapeutics, and vaccines. In one aspect, provided herein is an in vitro method for preparing APCs by culturing monocytes under conditions suitable for differentiation into immature dendritic cells (iDCs) and maturation of the (iDCs) into fully mature dendritic cells (mDCs). The monocytes and/or iDCs are contacted with one or more antigens, antigenic proteins, and/or libraries of antigen peptides under conditions suitable for internalization of such antigens, antigenic proteins and/or peptides by the monocytes and/or iDCs where at least a portion of the antigens, proteins and/or peptides will be subjected to proteolytic processing and ultimately presentation on the cell surface of the mDCs to produce APCs. As used herein, preloaded APCs refers to APCs that are produced from the differentiation of monocytes and/or iDCs that were loaded prior to full maturation with antigens, antigenic proteins, and/or peptides. Likewise, the terms “preload” and “preloading” are used in reference to the loading of monocytes and/or iDCs with antigens, antigenic proteins, and/or peptides. Certain embodiments of the present disclosure provide compositions comprising novel populations of APCs characterized by the presentation of the proteolytically processed antigens, proteins and/or peptides, as well as dendritic cell therapeutics and vaccines comprising such APCs. Also provided herein are compositions comprising novel populations of T cells primed or trained by such APCs, as well as T cell therapeutics comprising such T cells.

In some embodiments, the present disclosure provides a combination of conventionally loaded DCs and preloaded DCs. In some aspects, such combination may comprise a commixture of conventionally loaded DCs and preloaded DCs. Conventionally loaded DCs can comprise, or present on their surface (e.g., via MHC), a plurality of antigenic peptides having a uniform length, or having the same length(s) as the initial library of peptides provided to the DCs (e.g., mDCs) for loading. Preloaded DCs, on the other hand, can comprise, or present on their surface (e.g., via MHC), a plurality of peptides having different lengths (e.g., 2 different lengths, 3 different lengths, or more), each of which having been proteolytically processed and thus shortened in vivo by, e.g., monocytes or iDCs before presentation on the DCs. The range of lengths may vary greatly, depending on the full-length of the antigenic proteins or peptides—for example, if a peptide library of 15mers was used to preload the monocytes or iDCs, the population of DCs may present peptides ranging from 8 amino acids to 15 amino acids. Other aspects, such a combination comprises the use of conventionally loaded DCs and preloaded DCs separately in the same process, such as in ex vivo T cell priming in which conventionally loaded DCs are used to re-stimulate T cells that were first primed with preloaded DCs, or vice versa.

According to some embodiments, methods of the present disclosure may include the following steps:
(a) providing a plurality of monocytes;
(b) culturing a first aliquot of the monocytes in a first culture medium comprising cytokines (e.g., IL-4 and GMCSF), thereby inducing differentiation of at least a portion of the first aliquot of monocytes into immature dendritic cells (DCs);
(c) delivering to the monocytes and/or immature DCs a plurality of peptides (e.g., 15mers) derived from one or more tumor-associated antigens (TAAs) (“TAA peptides”), e.g., by incubation with the TAA peptides, whole TAA protein, or via peptide-conjugated liposomal delivery;
(d) continuing to culture the monocytes and/or immature DCs into a first plurality of mature DCs that present on their surfaces 6-15mer peptide antigens, preferably 8-11mer peptide antigens;
(e) culturing a second aliquot of the monocytes and/or a plurality of immature DCs in a second culture medium, thereby inducing differentiation into mature DCs;
(f) loading onto the mature DCs a plurality of the TAA peptides, thereby obtaining a second plurality of mature DCs that present on their surfaces the TAA peptides (e.g., 15mer peptides); and
(g) combining the first plurality of mature DCs and the second plurality of mature DCs at a ratio of about 10:1 to 1:10 (e.g., about 5:1 to 1:5, or about 1:1), thereby generating APCs suitable for downstream uses (e.g., T cell training).

In some embodiments, the immature DCs can be monocytes acquired by elutriating peripheral blood mononuclear cells (PBMCs) into at least a lymphocyte-rich fraction and a monocyte-rich fraction, wherein preferably the peripheral blood mononuclear cells are from a cancer patient in need of cell therapy.

In some embodiments, the peptides can include full-length TAAs and/or TAA fragments. The peptides can be a library of peptides obtained or derived from various TAAs. They can have a length of 8-15 amino acids (8-15mers). The TAAs can be, e.g., selected from PRAME, SSX2, NY-ESO-1, Survivin, and WT-1. In certain embodiments, the TAAs are obtained from the cancer patient in need of treatment. In certain embodiments, the TAAs can include viral tumor antigens for HPV⁺ head & neck cancer and/or cervical cancer.

The resulting APCs can display on their cell surface 8-10mer antigens presented by major histocompatibility complex (MHC) I, wherein the 8-10mers are created from antigens and/or peptides that are proteolytically processed by the monocytes and/or iDCs from the peptides.

In various embodiments, the APCs prepared in accordance with the methods disclosed herein can be used to expand multi-targeted T cells (MTCs) in vitro. This can be done by, e.g., co-culturing the lymphocyte-rich fraction of the PBMCs with the APCs to expand MTCs that are reactive to the TAA peptides. Such co-culturing can proceed in the presence of IL-15, IL-12, and optionally one or more of IL-21, IL-7, IL-2 and IL-6.

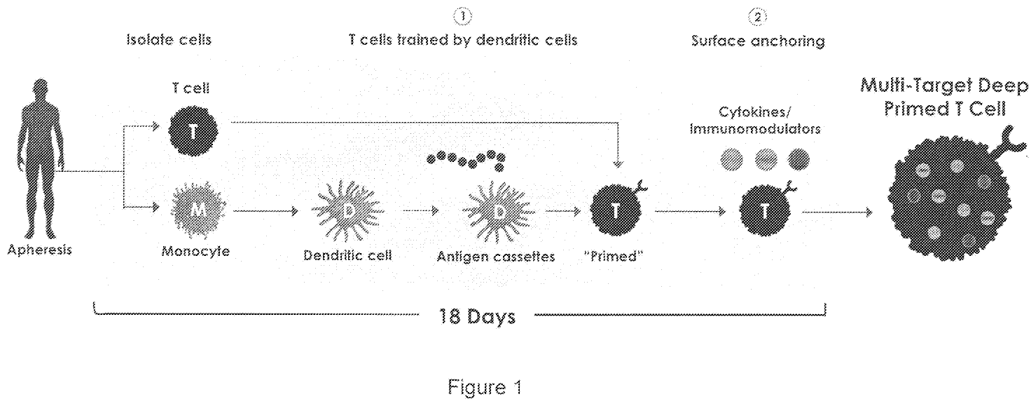

As shown in FIG. 1, the expanded MTCs can be loaded with clusters of therapeutic protein monomers to provide additional therapeutic benefits. Examples of therapeutic protein monomers include, without limitation, antibodies (e.g., IgG, Fab, mixed Fc and Fab), single chain antibodies, antibody fragments, engineered proteins such as Fc fusions, enzymes, co-factors, receptors, ligands, transcription factors and other regulatory factors, cytokines, chemokines, human serum albumin, and the like. These proteins may or may not be naturally occurring. Other proteins are contemplated and may be used in accordance with the disclosure. Any of the proteins can be reversibly modified through cross-linking to form a cluster or nanogel structure as disclosed in, e.g., U.S. Publication No. 2017/0080104, U.S. Pat. No. 9,603,944, U.S Publication No. 2014/0081012, PCT Application No. PCT/US17/37249 filed Jun. 13, 2017, and U.S. Provisional Application No. 62/657,218 filed Apr. 13, 2018, all incorporated herein by reference in their entirety. Loaded cells can have many therapeutic applications. For example, loaded MTCs can be used in T cell therapies including adoptive cell therapy.

### Definitions

Certain terms are defined herein below. Additional definitions are provided throughout the application.

As used herein, the articles “a” and “an” refer to one or more than one, e.g., to at least one, of the grammatical object of the article. The use of the words “a” or “an” when used in conjunction with the term “comprising” herein may mean “one,” but it is also consistent with the meaning of “one or more,” “at least one,” and “one or more than one.”

As used herein, “about” and “approximately” generally mean an acceptable degree of error for the quantity measured given the nature or precision of the measurements. Exemplary degrees of error are within 20 percent (%), typically, within 10%, and more typically, within 5% of a given range of values.

The term “autologous” refers to any material derived from the same individual to whom it is later to be re-introduced into the individual.

The term “allogeneic” refers to any material derived from a different animal of the same species as the individual to whom the material is introduced.

“Acquire” or “acquiring” or “obtain” or “obtaining” as the terms are used herein, refers to obtaining possession of a physical entity (e.g., a sample, a cell or cell population, a polypeptide, a nucleic acid, or a sequence), or a value, e.g., a numerical value, by “directly acquiring” or “indirectly acquiring” the physical entity or value. In one embodiment, acquiring refers to obtaining or harvesting a cell or cell population (e.g., an immune effector cell or population as described herein). “Directly acquiring” means performing a process (e.g., performing a synthetic or analytical or purification method) to obtain the physical entity or value. “Indirectly acquiring” refers to receiving the physical entity or value from another party or source (e.g., a third-party laboratory that directly acquired the physical entity or value).

“Immune cell,” as that term is used herein, refers to a cell that is involved in an immune response, e.g., in the promotion of an immune response. In some embodiments, the immune cell is an immune effector cell. Examples of immune effector cells include, but are not limited to, T cells, e.g., CD4+ and CD8+ T cells, alpha/beta T cells and gamma/delta T cells, B cells, natural killer (NK) cells, natural killer T (NKT) cells, and mast cells. “Immune cell” also refers to modified versions of cells involved in an immune response, e.g. modified NK cells, including NK cell line NK-92 (ATCC cat. No. CRL-2407), haNK (an NK-92 variant that expresses the high-affinity Fc receptor FcγRIIIa (158V)) and taNK (targeted NK-92 cells transfected with a gene that expresses a CAR for a given tumor antigen), e.g., as described in Klingemann et al. supra.

“Immune effector cell,” as that term is used herein, refers to a cell that is involved in an immune response, e.g., in the promotion of an immune effector response. Examples of immune effector cells include, but are not limited to, T cells, e.g., CD4+ T cells, CD8+ T cells, alpha T cells, beta T cells, gamma T cells, and delta T cells; B cells; natural killer (NK) cells; natural killer T (NKT) cells; dendritic cells; and mast cells. In some embodiments, the immune cell is an immune cell (e.g., T cell or NK cell) that comprises, e.g., expresses, a Chimeric Antigen Receptor (CAR), e.g., a CAR that binds to a cancer antigen. In other embodiments, the immune cell expresses an exogenous high affinity Fc receptor. In some embodiments, the immune cell comprises, e.g., expresses, an engineered T-cell receptor. In some embodiments, the immune cell is a tumor infiltrating lymphocyte. In some embodiments the immune cells comprise a population of immune cells and comprise T cells that have been enriched for specificity for a tumor-associated antigen (TAA), e.g., enriched by sorting for T cells with specificity towards MHCs displaying a TAA of interest, e.g. MART-1. In some embodiments immune cells comprise a population of immune cells and comprise T cells that have been “trained” to possess specificity against a TAA by an antigen presenting cell (APC), e.g., a dendritic cell, displaying TAA peptides of interest. In some embodiments, the T cells are trained against a TAA chosen from one or more of MART-1, MAGE-A4, NY-ESO-1, SSX2, Survivin, or others. In some embodiments the immune cells comprise a population of T cells that have been “trained” to possess specificity against multiple TAAs by an APC, e.g. a dendritic cell, displaying multiple TAA peptides of interest. Such T cells are also referred to as multi-targeted T cells (“MTC”) herein. In some embodiments, the immune cell is a cytotoxic T cell (e.g., a CD8+ T cell). In some embodiments, the immune cell is a helper T cell, e.g., a CD4+ T cell.

“Antigen-presenting cells (APCs)” are a group of immune cells that mnediate the cellular immune response by displaying antigen complexed to major histocompatibility complexes (MHCs) in the cell surface for recognition by certain lymphocytes such as T cells. Classical APCs include dendritic cells, macrophages, Langerhans cells and B cells.

The main function of “dendritic cells (DCs)” is to present antigens to T cells. Dendritic cells use two types of major histocompatibility complex (MHC) to display antigen peptides: MHC I and MHC II. MHC I trains CD8+ T-cells into cytotoxic, tumor-cell killers; and MHC II trains CD4+ T-cells into cytokine-producing helper cells. Clinical and pre-clinical data suggest both T-cell types help kill tumors. The peptides MHC I & MHC II present are not necessarily the same between each other nor between patients.

Immature dendritic cells (iDCs) are characterized by high endocytic activity and low T-cell activation potential. Immature dendritic cells phagocytose pathogens and degrade their proteins into small pieces and upon maturation present those fragments at their cell surface using MHC molecules. Simultaneously, they upregulate cell-surface receptors that act as co-receptors in T-cell activation such as CD80 (B7.1), CD86 (B7.2), and CD40, greatly enhancing their ability to activate T-cells. Once they have come into contact with a presentable antigen, they become activated into mature dendritic cells (mDCs), which in turn, activate helper T-cells and killer T-cells as well as B-cells by presenting them with antigens derived from the pathogen, alongside non-antigen specific costimulatory signals.

In some embodiments, dendritic cells can be generated in vivo (ex vitro) from monocytes, sometimes referred to as monocyte-derived dendritic cells. Briefly, the cells can transition from CD14+CD83− monocytes to CD14-CD83− immature DCs under the influence of IL-4 and GMCSF and then upregulate CD83 upon activation/maturation to become CD14-CD83+ mDC. See, e.g., Putz et al., Methods Mol Med. 2005; 109:71-82, incorporated herein by reference in its entirety. It should be noted that the transition from immature to mature DC is not instantaneous and requires some time, during which time the DCs are in a maturing process. Some DCs may mature faster than others and thus, the population may be a mix of immature, maturing, semi-mature, and mature DCs, while the population as a whole is in the process of maturing.

Monocyte-derived dendritic cells (moDC) can also be generated in vitro from peripheral blood mononuclear cells (PBMCs). Plating of PBMCs in a tissue culture flask permits adherence of monocytes. Treatment of these monocytes with interleukin 4 (IL-4) and granulocyte-macrophage colony stimulating factor (GM-CSF) leads to differentiation to immature dendritic cells. Subsequent treatment with tumor necrosis factor (TNF), IL6, IL1B, and/or PGE2 further differentiates the iDCs into mature DCs.

“Cytotoxic T lymphocytes” (CTLs) as used herein refer to T cells that have the ability to kill a target cell. CTL activation can occur when two steps occur: 1) an interaction between an antigen-bound MHC molecule on the target cell and a T cell receptor on the CTL is made; and 2) a costimulatory signal is made by engagement of costimulatory molecules on the T cell and the target cell. CTLs then recognize specific antigens on target cells and induce the destruction of these target cells, e.g., by cell lysis.

“Tumor infiltrating lymphocytes” (TILs) are used herein refer to lymphocytes that have migrated into a tumor. In embodiments, TILs can be cells at different stages of maturation or differentiation, e.g., TILs can include CTLs, Tregs, and/or effector memory T cells, among other types of lymphocytes.

“Tumor-associated antigen” (TAA) is an antigenic substance produced in tumor cells that triggers an immune response in the host. Tumor antigens are useful tumor markers in identifying tumor cells with diagnostic tests and are potential candidates for use in cancer therapy. In some embodiments, the TAA can be derived from, a cancer including but not limited to primary or metastatic melanoma, thymoma, lymphoma, sarcoma, lung cancer, liver cancer, non-Hodgkin's lymphoma, non-Hodgkins lymphoma, leukemias, uterine cancer, cervical cancer, bladder cancer, kidney cancer and adenocarcinomas such as breast cancer, prostate cancer, ovarian cancer, pancreatic cancer, and the like. TAAs can be patient specific. In some embodiments, TAAs may be p53, Ras, beta-Catenin, CDK4, alpha-Actinin-4, Tyrosinase, TRP1/gp75, TRP2, gplOO, Melan-A/MART 1, Gangliosides, PSMA, HER2, WT1, EphA3, EGFR, CD20, MAGE, BAGE, GAGE, NY-ESO-1, Telomerase, Survivin, or any combination thereof. Exemplary TAAs include preferentially expressed antigen of melanoma (PRAME), synovial sarcoma X (SSX) breakpoint 2 (SSX2), NY-ESO-1, Survivin, and Wilms' tumor gene 1 (WT-1).

The term “homologous” or “identity” refers to the subunit sequence identity between two polymeric molecules, e.g., between two nucleic acid molecules, such as, two DNA molecules or two RNA molecules, or between two polypeptide molecules. When a subunit position in both of the two molecules is occupied by the same monomeric subunit; e.g., if a position in each of two DNA molecules is occupied by adenine, then they are homologous or identical at that position. The homology between two sequences is a direct function of the number of matching or homologous positions; e.g., if half (e.g., five positions in a polymer of ten subunits in length) of the positions in two sequences are homologous, the two sequences are 50% homologous; if 90% of the positions (e.g., 9 of 10), are matched or homologous, the two sequences are 90% homologous.

The term “functional variant” in the context of a polypeptide refers to a polypeptide that is capable of having at least 10% of one or more activities of the naturally-occurring sequence. In some embodiments, the functional variant has substantial amino acid sequence identity to the naturally-occurring sequence, or is encoded by a substantially identical nucleotide sequence, such that the functional variant has one or more activities of the naturally-occurring sequence.

“Antibody molecule” as used herein refers to a protein, e.g., an immunoglobulin chain or fragment thereof, comprising at least one immunoglobulin variable domain sequence. An antibody molecule encompasses antibodies (e.g., full-length antibodies) and antibody fragments. For example, a full-length antibody is an immunoglobulin (Ig) molecule (e.g., an IgG antibody) that is naturally occurring or formed by normal immunoglobulin gene fragment recombinatorial processes). In embodiments, an antibody molecule refers to an immunologically active, antigen-binding portion of an immunoglobulin molecule, such as an antibody fragment. An antibody fragment, e.g., functional fragment, is a portion of an antibody, e.g., Fab, Fab′, F(ab′)2, F(ab)2, variable fragment (Fv), domain antibody (dAb), or single chain variable fragment (scFv). A functional antibody fragment binds to the same antigen as that recognized by the intact (e.g., full-length) antibody. The terms “antibody fragment” or “functional fragment” also include isolated fragments consisting of the variable regions, such as the “Fv” fragments consisting of the variable regions of the heavy and light chains or recombinant single chain polypeptide molecules in which light and heavy variable regions are connected by a peptide linker (“scFv proteins”). In some embodiments, an antibody fragment does not include portions of antibodies without antigen binding activity, such as Fc fragments or single amino acid residues. Exemplary antibody molecules include full length antibodies and antibody fragments, e.g., dAb (domain antibody), single chain, Fab, Fab′, and F(ab′)2 fragments, and single chain variable fragments (scFvs). In embodiments, an antibody molecule is monospecific, e.g., it comprises binding specificity for a single epitope. In some embodiments, an antibody molecule is multispecific, e.g., it comprises a plurality of immunoglobulin variable domain sequences, where a first immunoglobulin variable domain sequence has binding specificity for a first epitope and a second immunoglobulin variable domain sequence has binding specificity for a second epitope.

In some embodiments, an antibody molecule is a bispecific antibody molecule. “Bispecific antibody molecule” as used herein refers to an antibody molecule that has specificity for more than one (e.g., two, three, four, or more) epitope and/or antigen.

As used herein, “antigen” refers to a macromolecule, including all proteins or peptides. In some embodiments, an antigen is a molecule that can provoke an immune response, e.g., involving activation of certain immune cells and/or antibody generation. For the purpose of APC preparation, the antigen can be a full-length protein of one or more antigen or fragments larger that small peptides of one or more antigen. In some embodiments, the antigen can include disease antigens (e.g., tumor antigens, cell membrane antigens and extracellular matrix components). As used herein a “tumor antigen” or interchangeably, a “cancer antigen” includes any molecule present on, or associated with, a cancer, e.g., a cancer cell or a tumor microenvironment that can provoke an immune response. The tumor antigen may be a tumor associated antigen (TAA), a viral antigen, an antibody-recognized antigen, any fragment thereof, or any combination thereof.

As used herein, a “cytokine” or “cytokine molecule” refers to full length, a fragment or a variant of a naturally-occurring, wild type cytokine (including fragments and functional variants thereof having at least 10% of the activity of the naturally-occurring cytokine molecule). In embodiments, the cytokine molecule has at least 30, 50, or 80% of the activity, e.g., the immunomodulatory activity, of the naturally-occurring molecule. In embodiments, the cytokine molecule further comprises a receptor domain, e.g., a cytokine receptor domain, optionally, coupled to an immunoglobulin Fc region. In other embodiments, the cytokine molecule is coupled to an immunoglobulin Fc region. In other embodiments, the cytokine molecule is coupled to an antibody molecule (e.g., an immunoglobulin Fab or scFv fragment, a Fab fragment, a FAB2 fragment, or an affibody fragment or derivative, e.g. a sdAb (nanobody) fragment, a heavy chain antibody fragment, single-domain antibody, a bi-specific or multispecific antibody).

A “cytokine agonist,” as used herein can include an agonist of a cytokine receptor, e.g., an antibody molecule (e.g., an agonistic antibody) to a cytokine receptor, that elicits at least one activity of a naturally-occurring cytokine.

“Sample” or “tissue sample” refers to a biological sample obtained from a tissue or bodily fluid of a subject or patient. The source of the tissue sample can be solid tissue as from a fresh, frozen and/or preserved organ, tissue sample, biopsy, or aspirate; blood or any blood constituents (e.g., serum, plasma); bone marrow or any bone marrow constituents; bodily fluids such as urine, cerebral spinal fluid, whole blood, plasma and serum. The sample can include a non-cellular fraction (e.g., urine, plasma, serum, or other non-cellular body fluid). In other embodiments, the body fluid from which the sample is obtained from an individual comprises blood (e.g., whole blood).

The term “subject” includes living organisms in which an immune response can be elicited (e.g., mammals, human). In one embodiment, the subject is a patient, e.g., a patient in need of immune cell therapy. In another embodiment, the subject is a donor, e.g. an allogenic donor of immune cells, e.g., intended for allogenic transplantation.

The term, a “substantially purified cell” refers to a cell that is essentially free of other cell types and/or has been enriched relative to other cell types in the starting population. A substantially purified cell also refers to a cell which has been separated from other cell types with which it is normally associated in its naturally occurring state. In some instances, a population of substantially purified cells refers to a homogenous population of cells. In other instances, this term refers simply to cell that have been separated from the cells with which they are naturally associated in their natural state. In some aspects, the cells are cultured in vitro. In other aspects, the cells are not cultured in vitro.

Various aspects of the present disclosure may be used alone, in combination, or in a variety of arrangements not specifically discussed in the embodiments described in the foregoing and is therefore not limited in its application to the details and arrangement of components set forth in the foregoing description or illustrated in the drawings. For example, aspects described in one embodiment may be combined in any manner with aspects described in other embodiments.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for the use of the ordinal term) to distinguish the claim elements.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items. “Consisting essentially of” means inclusion of the items listed thereafter and which is open to unlisted items that do not materially affect the basic and novel properties of the disclosure.

### APC Preparation In Vitro

Antigen-presenting cells (APCs), e.g., dendritic cells (DCs) can be prepared in vitro using the methods disclosed herein. First, moDCs can be generated in vitro from peripheral blood mononuclear cells (PBMCs). Plating of PBMCs in a tissue culture flask permits adherence of monocytes. Treatment of these monocytes with interleukin 4 (IL-4) and granulocyte-macrophage colony stimulating factor (GM-CSF) leads to differentiation to iDCs. Subsequent treatment with tumor necrosis factor (TNF), IL6, IL1B, and PGE2 further differentiates the iDCs into mDCs.

Monocytes, iDCs and the cells prior to becoming mature DCs can be contacted with pre-selected antigens to be presented on their surface. This can be done in vitro using, in some embodiments, the preloading process disclosed herein. As used herein, preloading refers to a process where monocytes and/or immature DCs are induced to internalize and proteolytically process the peptides into shorter fragments for subsequent loading onto major histocompatibility complex (MHC) I and MHC II. The processed peptides may be stored by the monocytes and/or immature DCs for during the differentiation and/or maturation process and subsequently loaded onto the MHC by resulting mature DCs. Without wishing to be bound by theory, it is believed that most peptides loaded using the preloading process are 8mer-11mer in length (compared to standard initial peptides of 15mer). In contrast, the conventional process refers to the loading of TAA peptides onto previously matured DCs and is an extracellular method that briefly (typically for 1-3 hr) pulses DCs with peptide with the goal of loading peptides directly onto MHC I and MHC II at their original length without intracellular processing. This size difference between peptides loaded using preloading vs. conventional process is significant, because peptides that are presented in tumor MHC I are mostly shorter than 15mer (typically 8-10mer). As such, CD8+ CTLs that are trained by the conventional (i.e., extracellular loading) method using 15mer cannot be expected to bind tumor peptide:MHC due to intrinsic biophysical differences between loading of short (8-10mer) and long (15mer) peptides. Preloading uses intracellular processing of peptides to present peptides that are MHC I allele-specific and thus, can result in a more robust stimulation of a physiologically relevant CTL repertoire that can bind tumor peptide:MHC better and more effectively. Furthermore, using preloading, the peptides may be customized by the cell via proteolysis (which may be different across patients), so that the most biologically preferred peptides are loaded regardless of MHC allele. In various embodiments, disclosed herein is a combination composition (for example, a mixture of conventionally loaded DCs and preloaded DCs) and methods for making and using the same.

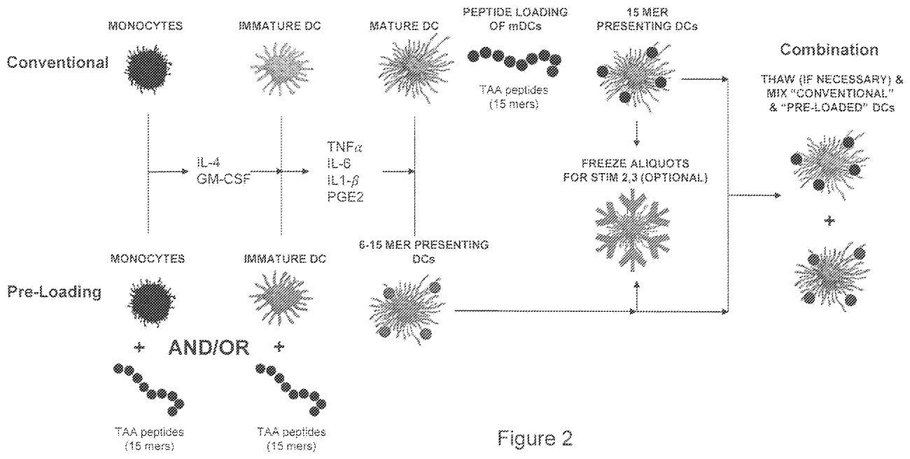

In some embodiments, an APC preparation method of the present disclosure can include the following steps (FIG. 2):
(a) providing a plurality of monocytes;
(b) culturing a first aliquot of the monocytes in a first culture medium comprising cytokines (e.g., IL-4 and GMCSF), thereby inducing differentiation of at least a portion of the first aliquot of monocytes into immature dendritic cells (DCs);
(c) delivering to the monocytes and/or immature DCs a plurality of peptides (e.g., 15mers) derived from one or more tumor-associated antigens (TAAs), e.g., by incubation with the TAA peptides, whole TAA protein, or via peptide-conjugated liposomal delivery;
(d) continuing to culture the monocytes and/or immature DCs into a first plurality of mature DCs that present on their surfaces 6-15mer peptide antigens, preferably 8-11mer peptide antigens;
(e) culturing a second aliquot of the monocytes and/or a plurality of immature DCs in a second culture medium, thereby inducing differentiation into mature DCs;
(f) loading onto the mature DCs a plurality of the TAA peptides, thereby obtaining a second plurality of mature DCs that present on their surfaces the TAA peptides (e.g., 15mer peptides); and
(g) combining the first plurality of mature DCs and the second plurality of mature DCs at a ratio of about 10:1 to 1:10 (e.g., about 5:1 to 1:5, or about 1:1), thereby generating APCs suitable for downstream uses (e.g., T cell training).

In some embodiments, the monocytes can be acquired by elutriating PBMCs into at least a lymphocyte-rich fraction and a monocyte-rich fraction, wherein preferably the PBMCs are from a cancer patient in need of cell therapy.

In some embodiments, the peptides can include full-length TAAs and/or TAA fragments. The peptides can be a library of peptides obtained or derived from various TAAs. They can have a length of 8-15 amino acids (8-15mers). The TAAs can be, e.g., selected from PRAME, SSX2, NY-ESO-1, Survivin, and WT-1. In certain embodiments, the TAAs are obtained from the cancer patient in need of treatment. In certain embodiments, the TAAs can include viral tumor antigens for HPV⁺ head & neck cancer and/or cervical cancer.

The resulting APCs can display on their cell surface 8-10mer antigens presented by major histocompatibility complex (MHC) I, wherein the 8-10mers are created from antigens and/or peptides that are proteolytically processed by the monocytes and/or iDCs from the peptides.

### MTC Preparation In Vitro

In various embodiments, the APCs prepared in accordance with the methods disclosed herein can be used to expand multi-targeted T cells (MTCs) in vitro. This can be done by, e.g., co-culturing the lymphocyte-rich fraction of the PBMCs with the APCs (e.g., at a ratio between about 40:1 to about 1:1) to expand MTCs that are reactive to the TAA peptides. Such co-culturing can proceed in the presence of one or more of IL-2, IL-6, IL-7, IL-12, IL-15 and IL-21. In some embodiments, co-culturing can be in the presence of IL-15, IL-12 and optionally one or more of IL-2, IL-21, IL-7 and IL-6. Advantageously, using methods and compositions disclosed herein, the entire process time from PBMCs to MTCs can be shortened to 10-20 days, whereas conventional methods typically require at least 20 days (see, e.g., Putz et al., Methods Mol Med. 2005; 109:71-82, incorporated herein by reference in its entirety). The resulting MTCs can be used in various T-cell therapies as further disclosed herein.

### Cytokine Molecules

The expanded MTCs can be loaded with clusters of therapeutic protein monomers (Deep Primed™) to provide additional therapeutic benefits. Examples of therapeutic protein monomers include, without limitation, antibodies (e.g., IgG, Fab, mixed Fc and Fab), single chain antibodies, antibody fragments, engineered proteins such as Fc fusions, enzymes, co-factors, receptors, ligands, transcription factors and other regulatory factors, cytokines, chemokines, human serum albumin, and the like. These proteins may or may not be naturally occurring. Other proteins are contemplated and may be used in accordance with the disclosure. Any of the proteins can be reversibly modified through cross-linking to form a cluster or nanogel structure as disclosed in, e.g., U.S. Publication No. 2017/0080104, U.S. Pat. No. 9,603,944, U.S Publication No. 2014/0081012, PCT Application No. PCT/US17/37249 filed Jun. 13, 2017, and U.S. Provisional Application No. 62/657,218 filed Apr. 13, 2018, all incorporated herein by reference in their entirety. Loaded cells can have many therapeutic applications. For example, loaded MTCs can be used in T cell therapies including adoptive cell therapy.

The therapeutic protein monomers can include one or more cytokine molecules. In embodiments, the cytokine molecule is full length, a fragment or a variant of a cytokine, e.g., a cytokine comprising one or more mutations. In some embodiments the cytokine molecule comprises a cytokine chosen from interleukin-1 alpha (IL-1 alpha), interleukin-1 beta (IL-1 beta), interleukin-2 (IL-2), interleukin-4 (IL-4), interleukin-5 (IL-5), interleukin-6 (IL-6), interleukin-7 (IL-7), interleukin-12 (IL-12), interleukin-15 (IL-15), interleukin-17 (IL-17), interleukin-18 (IL-18), interleukin-21 (IL-21), interleukin-23 (IL-23), interferon (IFN) alpha, IFN beta, IFN gamma, tumor necrosis alpha, GM-CSF, GCSF, or a fragment or variant thereof, or a combination of any of the aforesaid cytokines. In other embodiments, the cytokine molecule is chosen from interleukin-2 (IL-2), interleukin-7 (IL-7), interleukin-12 (IL-12), interleukin-15 (IL-15), interleukin-18 (IL-18), interleukin-21 (IL-21), interleukin-23 (IL-23) or interferon gamma, or a fragment or variant thereof, or a combination of any of the aforesaid cytokines. The cytokine molecule can be a monomer or a dimer.

In embodiments, the cytokine molecule further comprises a receptor domain, e.g., a cytokine receptor domain. In one embodiment, the cytokine molecule comprises an IL-15 receptor, or a fragment thereof (e.g., an extracellular IL-15 binding domain of an IL-15 receptor alpha) as described herein. In some embodiments, the cytokine molecule is an IL-15 molecule, e.g., IL-15 or an IL-15 superagonist as described herein. As used herein, a superagonist form of a cytokine molecule shows increased activity, e.g., by at least 10%, 20%, 30%, compared to the naturally-occurring cytokine. An exemplary superagonist is an IL-15 SA. In some embodiments, the IL-15 SA comprises a complex of IL-15 and an IL-15 binding fragment of an IL-15 receptor, e.g., IL-15 receptor alpha or an IL-15 binding fragment thereof.

In other embodiments, the cytokine molecule further comprises an antibody molecule, e.g., an immunoglobulin Fab or scFv fragment, a Fab fragment, a FAB2 fragment, or an affibody fragment or derivative, e.g., a sdAb (nanobody) fragment, a heavy chain antibody fragment, e.g., an Fc region, single-domain antibody, a bi-specific or multispecific antibody). In one embodiment, the cytokine molecule further comprises an immunoglobulin Fc or a Fab.

In some embodiments, the cytokine molecule is an IL-2 molecule, e.g., IL-2 or IL-2-Fc. In other embodiments, a cytokine agonist can be used in the methods and compositions disclosed herein. In embodiments, the cytokine agonist is an agonist of a cytokine receptor, e.g., an antibody molecule (e.g., an agonistic antibody) to a cytokine receptor, that elicits at least one activity of a naturally-occurring cytokine. In embodiments, the cytokine agonist is an agonist of a cytokine receptor, e.g., an antibody molecule (e.g., an agonistic antibody) to a cytokine receptor chosen from an IL-15Ra or IL-21R.

Exemplary cytokines are disclosed in PCT Application No. PCT/US17/37249, incorporated herein by reference in their entirety.

### Therapeutic Uses and Methods

The preloaded DCs, mDCs, combination DCs, APCs, MTCs, and pharmaceutical compositions containing any of the foregoing have numerous therapeutic utilities, including, e.g., the treatment of cancers, autoimmune disorders and infectious diseases. The compositions can also be used in vaccine applications. They can be useful for ex vivo preparation of a cell therapy such as an adoptive cell therapy, CAR-T cell therapy, engineered TCR T cell therapy, a tumor infiltrating lymphocyte therapy, an antigen-trained T cell therapy, an enriched antigen-specific T cell therapy, or an NK cell therapy. The various dendritic cell compositions also can be transferred as a DC therapy for vaccination or cancer therapies.

In some embodiments, the present disclosure provides, inter alia, methods for inducing an immune response in a subject with a cancer in order to treat the subject having cancer. Exemplary methods comprise administering to the subject a therapeutically effective amount of any of the compositions described herein.

Methods described herein include treating a cancer in a subject by using any of the compositions disclosed herein. Also provided are methods for reducing or ameliorating a symptom of a cancer in a subject, as well as methods for inhibiting the growth of a cancer and/or killing one or more cancer cells. In embodiments, the methods described herein decrease the size of a tumor and/or decrease the number of cancer cells in a subject administered with a described herein or a pharmaceutical composition described herein.

In embodiments, the cancer is a hematological cancer. In embodiments, the hematological cancer is a leukemia or a lymphoma. As used herein, a “hematologic cancer” refers to a tumor of the hematopoietic or lymphoid tissues, e.g., a tumor that affects blood, bone marrow, or lymph nodes. Exemplary hematologic malignancies include, but are not limited to, leukemia (e.g., acute lymphoblastic leukemia (ALL), acute myeloid leukemia (AML), chronic lymphocytic leukemia (CLL), chronic myelogenous leukemia (CML), hairy cell leukemia, acute monocytic leukemia (AMoL), chronic myelomonocytic leukemia (CMML), juvenile myelomonocytic leukemia (JMML), or large granular lymphocytic leukemia), lymphoma (e.g., AIDS-related lymphoma, cutaneous T-cell lymphoma, Hodgkin lymphoma (e.g., classical Hodgkin lymphoma or nodular lymphocyte-predominant Hodgkin lymphoma), mycosis fungoides, non-Hodgkin lymphoma (e.g., B-cell non-Hodgkin lymphoma (e.g., Burkitt lymphoma, small lymphocytic lymphoma (CLL/SLL), diffuse large B-cell lymphoma, follicular lymphoma, immunoblastic large cell lymphoma, precursor B-lymphoblastic lymphoma, or mantle cell lymphoma) or T-cell non-Hodgkin lymphoma (mycosis fungoides, anaplastic large cell lymphoma, or precursor T-lymphoblastic lymphoma)), primary central nervous system lymphoma, Sezary syndrome, Waldenström macroglobulinemia), chronic myeloproliferative neoplasm, Langerhans cell histiocytosis, multiple myeloma/plasma cell neoplasm, myelodysplastic syndrome, or myelodysplastic/myeloproliferative neoplasm.

In embodiments, the cancer is a solid cancer. Exemplary solid cancers include, but are not limited to, ovarian cancer, rectal cancer, stomach cancer, testicular cancer, cancer of the anal region, uterine cancer, colon cancer, rectal cancer, renal-cell carcinoma, liver cancer, non-small cell carcinoma of the lung, cancer of the small intestine, cancer of the esophagus, melanoma, Kaposi's sarcoma, cancer of the endocrine system, cancer of the thyroid gland, cancer of the parathyroid gland, cancer of the adrenal gland, bone cancer, pancreatic cancer, skin cancer, cancer of the head or neck, cutaneous or intraocular malignant melanoma, uterine cancer, brain stem glioma, pituitary adenoma, epidermoid cancer, carcinoma of the cervix squamous cell cancer, carcinoma of the fallopian tubes, carcinoma of the endometrium, carcinoma of the vagina, sarcoma of soft tissue, cancer of the urethra, carcinoma of the vulva, cancer of the penis, cancer of the bladder, cancer of the kidney or ureter, carcinoma of the renal pelvis, spinal axis tumor, neoplasm of the central nervous system (CNS), primary CNS lymphoma, tumor angiogenesis, metastatic lesions of said cancers, or combinations thereof.

In embodiments, the cell compositions (or pharmaceutical composition containing the same) are administered in a manner appropriate to the disease to be treated or prevented. The quantity and frequency of administration will be determined by such factors as the condition of the patient, and the type and severity of the patient's disease. Appropriate dosages may be determined by clinical trials. For example, when “an effective amount” or “a therapeutic amount” is indicated, the precise amount of the pharmaceutical composition to be administered can be determined by a physician with consideration of individual differences in tumor size, extent of infection or metastasis, age, weight, and condition of the subject. In embodiments, the pharmaceutical composition described herein can be administered at a dosage of 10⁴ to 10⁹ cells/kg body weight, e.g., 10⁵ to 10⁶ cells/kg body weight, including all integer values within those ranges. In embodiments, the pharmaceutical composition described herein can be administered multiple times at these dosages. In embodiments, the pharmaceutical composition described herein can be administered using infusion techniques described in immunotherapy (see, e.g., Rosenberg et al., New Eng. J. of Med. 319:1676, 1988).

In embodiments, the pharmaceutical composition is administered to the subject parenterally. In embodiments, the cells are administered to the subject intravenously, subcutaneously, intratumorally, intranodally, intramuscularly, intradermally, or intraperitoneally. In embodiments, the cells are administered, e.g., injected, directly into a tumor or lymph node. In embodiments, the cells are administered as an infusion (e.g., as described in Rosenberg et al., New Eng. J. of Med. 319:1676, 1988) or an intravenous push. In embodiments, the cells are administered as an injectable depot formulation.

In embodiments, the subject is a mammal. In embodiments, the subject is a human, monkey, pig, dog, cat, cow, sheep, goat, rabbit, rat, or mouse. In embodiments, the subject is a human. In embodiments, the subject is a pediatric subject, e.g., less than 18 years of age, e.g., less than 17, 16, 15, 14, 13, 12, 11, 10, 9, 8, 7, 6, 5, 4, 3, 2, 1 or less years of age. In embodiments, the subject is an adult, e.g., at least 18 years of age, e.g., at least 19, 20, 21, 22, 23, 24, 25, 25-30, 30-35, 35-40, 40-50, 50-60, 60-70, 70-80, or 80-90 years of age.

### Combination Therapies

The cell compositions disclosed herein can be used in combination with a second therapeutic agent or procedure, such as surface loading or co-administration with one or more immunomodulating cytokines disclosed herein.

In some embodiments, the cell composition is administered in combination with radiotherapy.

In embodiments, the cell composition and the second therapeutic agent or procedure are administered/performed after a subject has been diagnosed with a cancer, e.g., before the cancer has been eliminated from the subject. In embodiments, the cell composition and the second therapeutic agent or procedure are administered/performed simultaneously or concurrently. For example, the delivery of one treatment is still occurring when the delivery of the second commences, e.g., there is an overlap in administration of the treatments. In other embodiments, the cell composition and the second therapeutic agent or procedure are administered/performed sequentially. For example, the delivery of one treatment ceases before the delivery of the other treatment begins.

In embodiments, combination therapy can lead to more effective treatment than monotherapy with either agent alone. In embodiments, the combination of the first and second treatment is more effective (e.g., leads to a greater reduction in symptoms and/or cancer cells) than the first or second treatment alone. In embodiments, the combination therapy permits use of a lower dose of the first or the second treatment compared to the dose of the first or second treatment normally required to achieve similar effects when administered as a monotherapy. In embodiments, the combination therapy has a partially additive effect, wholly additive effect, or greater than additive effect.

In one embodiment, the cell composition is administered in combination with a therapy, e.g., a cancer therapy (e.g., one or more of anti-cancer agents, immunotherapy, photodynamic therapy (PDT), surgery and/or radiation). The terms “chemotherapeutic,” “chemotherapeutic agent,” and “anti-cancer agent” are used interchangeably herein. The administration of the cell composition and the therapy, e.g., the cancer therapy, can be sequential (with or without overlap) or simultaneous. Administration of the cell composition can be continuous or intermittent during the course of therapy (e.g., cancer therapy). Certain therapies described herein can be used to treat cancers and non-cancerous diseases. For example, PDT efficacy can be enhanced in cancerous and non-cancerous conditions (e.g., tuberculosis) using the methods and compositions described herein (reviewed in, e.g., Agostinis, P. et al. (2011) CA Cancer J Clin. 61:250-281).

In other embodiments, the cell composition is administered in combination with a low or small molecular weight chemotherapeutic agent. Exemplary low or small molecular weight chemotherapeutic agents include, but not limited to, 13-cis-retinoic acid (isotretinoin, ACCUTANE®), 2-CdA (2-chlorodeoxyadenosine, cladribine, LEUSTATIN™), 5-azacitidine (azacitidine, VIDAZA®), 5-fluorouracil (5-FU, fluorouracil, ADRUCIL®), 6-mercaptopurine (6-MP, mercaptopurine, PURINETHOL®), 6-TG (6-thioguanine, thioguanine, THIOGUANINE TABLOID®), abraxane (paclitaxel protein-bound), actinomycin-D (dactinomycin, COSMEGEN®), alitretinoin (PANRETIN®), all-transretinoic acid (ATRA, tretinoin, VESANOID®), altretamine (hexamethylmelamine, HMM, HEXALEN®), amethopterin (methotrexate, methotrexate sodium, MTX, TREXALL™, RHEUMATREX®), amifostine (ETHYOL®), arabinosylcytosine (Ara-C, cytarabine, CYTOSAR-U®), arsenic trioxide (TRISENOX®), asparaginase (Erwinia L-asparaginase, L-asparaginase, ELSPAR®, KIDROLASE®), BCNU (carmustine, BiCNU®), bendamustine (TREANDA®), bexarotene (TARGRETIN®), bleomycin (BLENOXANE®), busulfan (BUSULFEX®, MYLERAN®), calcium leucovorin (Citrovorum Factor, folinic acid, leucovorin), camptothecin-11 (CPT-11, irinotecan, CAMPTOSAR®), capecitabine (XELODA®), carboplatin (PARAPLATIN®), carmustine wafer (prolifeprospan 20 with carmustine implant, GLIADEL® wafer), CCI-779 (temsirolimus, TORISEL®), CCNU (lomustine, CeeNU), CDDP (cisplatin, PLATINOL®, PLATINOL-AQ®), chlorambucil (leukeran), cyclophosphamide (CYTOXAN®, NEOSAR®), dacarbazine (DIC, DTIC, imidazole carboxamide, DTIC-DOME®), daunomycin (daunorubicin, daunorubicin hydrochloride, rubidomycin hydrochloride, CERUBIDINE®), decitabine (DACOGEN®), dexrazoxane (ZINECARD®), DHAD (mitoxantrone, NOVANTRONE®), docetaxel (TAXOTERE®), doxorubicin (ADRIAMYCIN®, RUBEX®), epirubicin (ELLENCE™), estramustine (EMCYT®), etoposide (VP-16, etoposide phosphate, TOPOSAR®, VEPESID®, ETOPOPHOS®), floxuridine (FUDR®), fludarabine (FLUDARA®), fluorouracil (cream) (CARAC™, EFUDEX®, FLUOROPLEX®), gemcitabine (GEMZAR®), hydroxyurea (HYDREA®, DROXIA™, MYLOCEL™), idarubicin (IDAMYCIN®), ifosfamide (IFEX®), ixabepilone (IXEMPRA™), LCR (leurocristine, vincristine, VCR, ONCOVIN®, VINCASAR PFS®), L-PAM (L-sarcolysin, melphalan, phenylalanine mustard, ALKERAN®), mechlorethamine (mechlorethamine hydrochloride, mustine, nitrogen mustard, MUSTARGEN®), mesna (MESNEX™), mitomycin (mitomycin-C, MTC, MUTAMYCIN®), nelarabine (ARRANON®), oxaliplatin (ELOXATIN™), paclitaxel (TAXOL®, ONXAL™), pegaspargase (PEG-L-asparaginase, ONCOSPAR®), PEMETREXED (ALIMTA®), pentostatin (NIPENT®), procarbazine (MATULANE®), streptozocin (ZANOSAR®), temozolomide (TEMODAR®), teniposide (VM-26, VUMON®), TESPA (thiophosphoamide, thiotepa, TSPA, THIOPLEX®), topotecan (HYCAMTIN®), vinblastine (vinblastine sulfate, vincaleukoblastine, VLB, ALKABAN-AQ®, VELBAN®), vinorelbine (vinorelbine tartrate, NAVELBINE®), and vorinostat (ZOLINZA®).

In another embodiment, cell composition is administered in conjunction with a biologic. Exemplary biologics include, e.g., HERCEPTIN® (trastuzumab); FASLODEX® (fulvestrant); ARIMIDEX® (anastrozole); Aromasin® (exemestane); FEMARA® (letrozole); NOLVADEX® (tamoxifen), AVASTIN® (bevacizumab); and ZEVALIN® (ibritumomab tiuxetan).

### EXAMPLES

### Example 1: Combination Process Overview

An exemplary “combination” method consisting of combining a preloading method with a conventional loading method is shown in FIG. 2. Briefly, in the conventional method, monocytes are first treated with interleukin 4 (IL-4) and granulocyte-macrophage colony stimulating factor (GM-CSF) to induce differentiation to iDCs. Subsequent treatment with tumor necrosis factor (TNF□), IL-6, IL-1□, and PGE2 differentiates the iDCs into mDCs. 15mer TAA peptides are then added to the mDCs for direct loading producing a population containing 15mer-presenting DCs. The mature DCs produced via the conventional method can optionally be frozen for later use. When ready to use (e.g., in cell therapy), previously frozen aliquots of DCs can be thawed or used fresh in combination with DCs from the preloading method described below.

In the preloading method, 15mer TAA peptides are first added to monocytes and/or iDCs (as opposed to mDCs). After differentiation to mature DCs, 6-15mer, preferably 8-11mer-presenting DCs are obtained. These 6-15mer presenting mDCs can be optionally frozen, directly combined with 15mer-presenting mDCs obtained from the conventional method (e.g., at 1:1 ratio) to create a combination of DCs or used alone. The preloaded, conventional, or combination DCs can be frozen for later use. When ready to use (e.g., in cell therapy), fresh DCs or frozen aliquots can be thawed for co-culture with T cells.

### Example 2: Preloading Process Proof of Concept Using MART1 15Mer

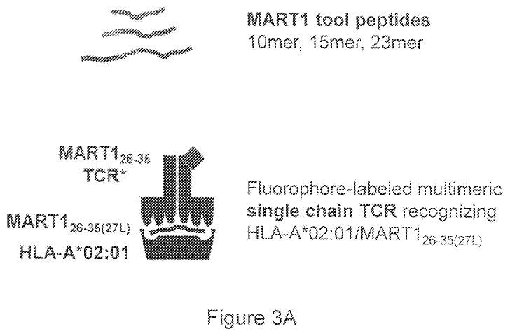

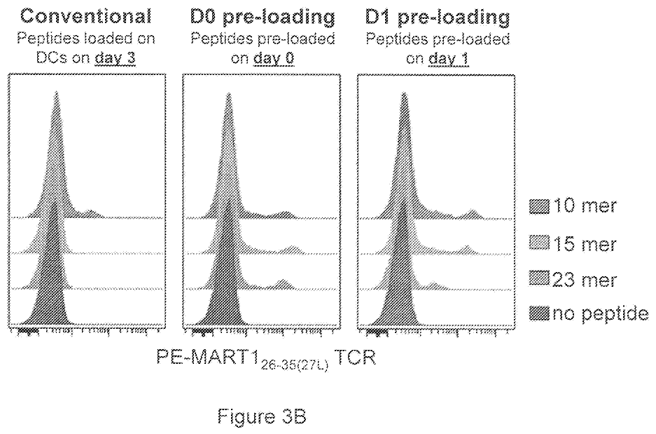

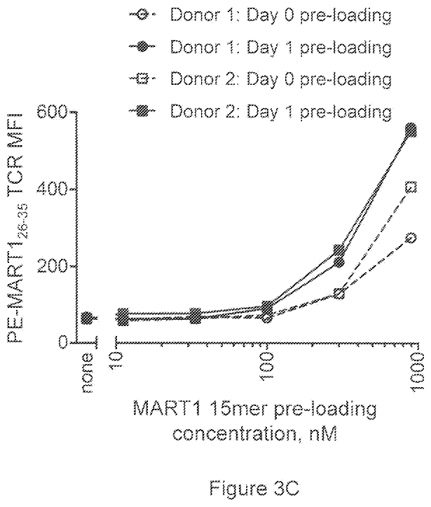

MART1 is a tumor-associated antigen expressed by some melanoma tumors. The MART1 protein contains an immunogenic peptide at positions 26-35 that is restricted to the HLA-A*02:01 MHC I allele. The encoded 10mer peptide ELAGIGILTV (SEQ ID NO: 1), which contains a leucine substitution at position 27 to enhance affinity for HLA-A*02:01, is a prototypical antigen for evaluating priming response in human T cells. In this proof-of-concept (POC) experiment (FIG. 3), dendritic cells are loaded with overlapping MART1 peptides (10mer, 15mer, or 23mer) using either the conventional loading (1 hour exposure of mature DCs to peptides) method or preloading (continuous exposure to peptides during differentiation and maturation of monocytes to mature dendritic cells) method. All peptides contain the 10mer: MART_{26-35(27L)}. The dendritic cells are labeled with a soluble single chain HLA-A*02:01/MART_{126-35(27L)}-specific TCR to test peptide:MHC I presentation (FIG. 3A).

As shown in FIG. 3B, when the conventional loading method is used, the TCR only recognizes presentation of the 10mer. In contrast, the TCR recognizes the processed 10mer epitope version of the 10mer, 15mer, or 23mer when peptides are preloaded on day 0 (preloaded as monocytes) or day 1 (preloaded as immature DCs) of DC generation. Thus, this POC experiment confirms that preloading of DCs results in processing and presentation of longer peptides into biologically appropriate MHC I epitopes.

As shown in FIG. 3C, preloading of the MART1 15mer peptide on both day 0 and day 1 of DC generation results in dose-responsive presentation of 10mer peptide:MHC I complexes across a range of 15mer peptide doses as detected by the MART-specific TCR.

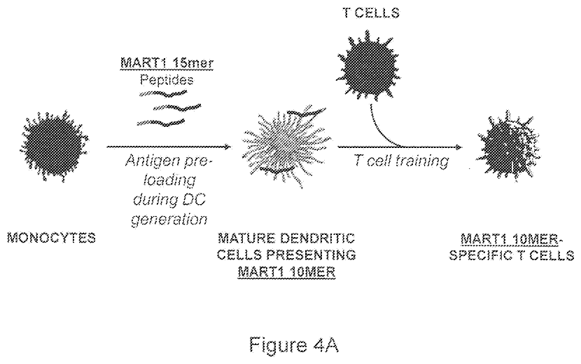

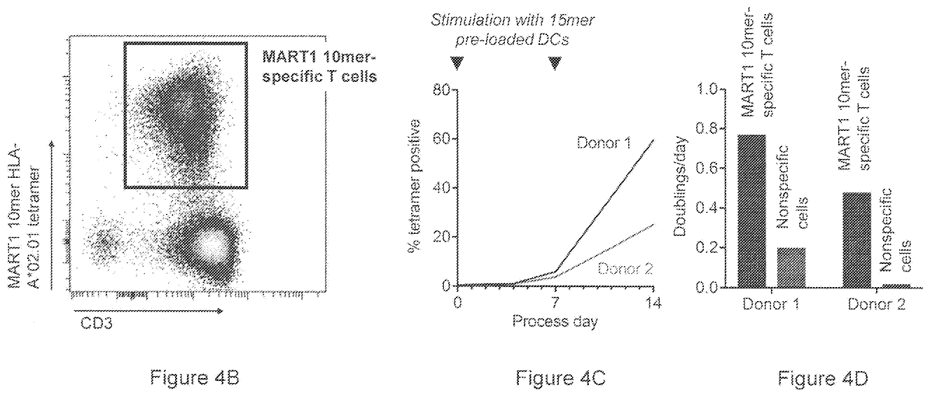

FIG. 4A illustrates the strategy used to validate functionality of 15mer preloaded dendritic cells for use in priming of MART1 10mer-specific T cells.

MART1 10mer-specific T cells can be identified by labeling of their TCRs with peptide:MHC tetramer reagents (FIG. 4B).

Using the scheme shown in FIG. 4A, autologous T cells are stimulated with MART1 15mer preloaded DCs at a ratio of 10:1 T cells:DC on day 0 and day 7 of a 14-day T cell training co-culture. As shown in FIG. 3C, these 15mer preloaded DCs present MART1 10mer in the context of HLA-A*02:01, which is the target peptide:MHC epitope for a subset (˜1 in 1000 naïve T cells) of CD8+ T cells. In FIG. 4C, enrichment of MART1 10mer-specific T cells is shown for two healthy HLA-A*02:01 donors. FIG. 4D illustrates that MART1 15mer preloaded DCs drive preferential expansion of MART1 10mer-specific T cells over nonspecific bystander T cells. Stimulation with non-loaded DCs results in no expansion or enrichment of MART1-specific cells (data not shown).

### Example 3: Conventionally Loaded, Preloaded, and Combination of Conventional/Preloaded Dendritic Cells for MTC Training

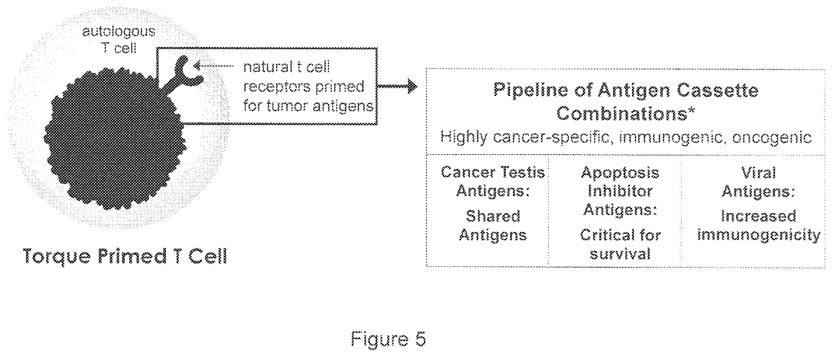

In vivo, antigenic peptides are processed and presented in a unique DC-dependent manner (i.e., proteolysis preferences and MHC haplotype are different from person to person). The preloading method takes advantage of this customization by allowing monocytes and/or iDCs to internalize, proteolytically process and subsequently load preferred peptides onto the MHC (as exemplified for MART1 in FIG. 3). However, this method can be expanded to include diversified libraries of peptides (FIG. 5). The technique expands the library of what is presented to T cells as well as ensures that peptides that are able to be presented on a patient's DCs are accessible for presentation from the 15mer library. Additionally, shorter 8-15mer peptides are better able to be loaded onto MHC I and are likely to better engage MTC.

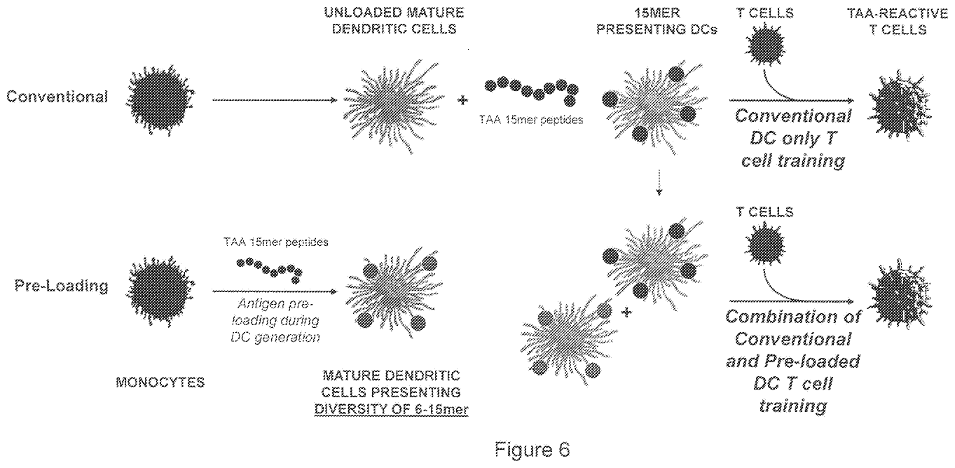

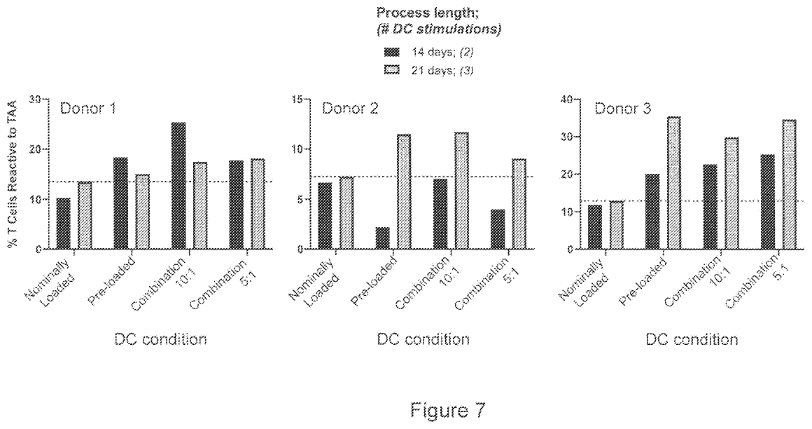

A scheme to evaluate the ability of conventionally loaded DCs and preloaded DCs to train TAA-reactive multi-targeted T cells (MTC) is shown in FIG. 6. Since the stimulation and expansion of a diverse pool of TAA-reactive, tumor-targeted T cells is favorable for immune cell therapy, combinations of conventionally loaded DCs and preloaded DCs (as illustrated in FIG. 2), which would be expected to show high peptide-MHC diversity, were additionally compared to conventionally loaded DCs. Peptides used for loading include off-the-shelf 15mer peptide pools from PRAME, WT-1, Survivin, NY-ESO-1, and SSX-2. For 3 healthy donors, T cell-enriched autologous PBMCs were stimulated on days 0 and 7 (“14 days”) or on days 0, 7, and 14 (“21 days”) using TAA-loaded DCs: (a) conventionally loaded at 10:1 T cell:DC; (b) preloaded at 10:1 T cell:DC; (c) a 1:1 combination of conventionally loaded DCs and preloaded DCs at 10:1 T cell:total DC; (d) a 1:1 combination of conventionally loaded DCs and preloaded DCs at 5:1 T cell:total DC. Upon harvest on day 14 or day 21, MTC are co-cultured overnight with TAA-loaded and unloaded DCs to assess TAA-specific reactivity, which is computed as the difference in percent activated T cells between antigen-loaded and unloaded co-cultures. Variability among donors was expectedly high, but all DC conditions enrich TAA-reactive T cells (FIG. 7). Generally, 21-day cultures show increased reactivity versus 14-day cultures due to a third stimulation. For 3/3 donors, reactivity by day 14 in cultures trained using a combination of conventional and preloaded DCs (10:1) was greater than or equivalent to reactivity achieved by day 21 in cultures trained using conventionally loaded DCs.

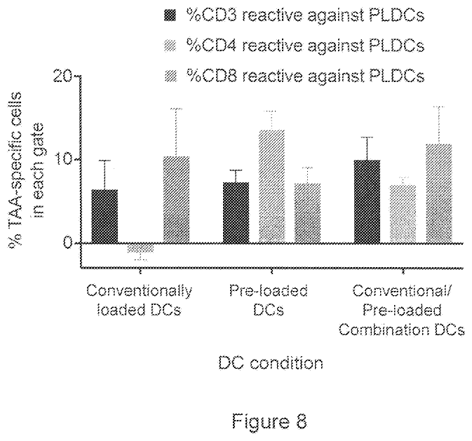

For one donor, TAA reactivity in the CD3, CD4, and CD8 T cell compartments was compared, revealing that different TAA loading strategies can result in different T cell reactivity signatures (FIG. 8). Briefly, training T cells with conventionally loaded DCs resulted in TAA reactivity exclusively in the CD8 compartment, whereas training T cells with preloaded DCs or a combination of conventional and preloaded DCs led to TAA reactivity in both CD4 and CD8 T cells. These results indicate that increased diversity of antigen presentation by DCs used for ex vivo T cell training could lead to increased diversity of the resulting T cell product.

A modification of the scheme shown in FIG. 6 was used to directly evaluate the ability of conventionally loaded DCs and preloaded DCs to train different TAA-reactive MTCs. Briefly, a mixture of off-the-shelf 15mer peptide pools from PRAME, WT-1, and Survivin was supplemented with equimolar MART1 15mer. Since MART1 10mer-specific T cells can be reproducibly expanded from HLA-A*02:01 individuals when stimulated with MART1 10mer-bearing APCs, this strategy sought to use MART1 10mer-specific cells as a surrogate readout for reactivity diversity.

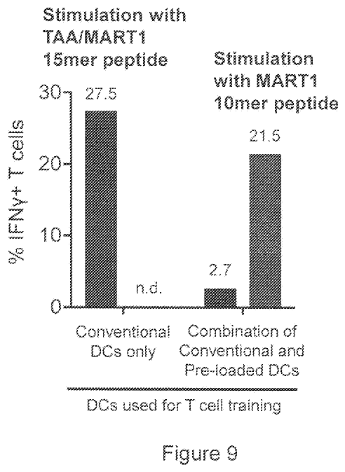

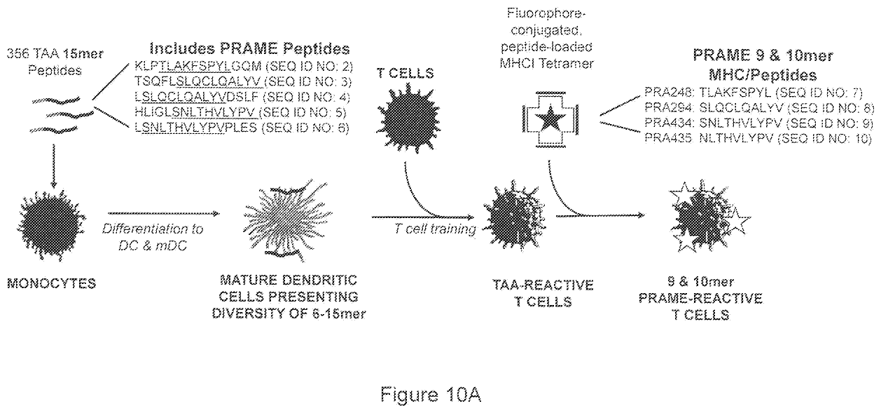

Following expansion of TAA-reactive T cells using either Conventional DCs only or a combination of conventionally loaded DCs and preloaded DCs, T cells were stimulated with TAA/MART1 15mer peptides or the MART1 10mer and the T cell activation response was assessed using flow cytometry analysis of IFN-γ production (FIG. 9). T cells trained using conventionally loaded DCs only showed reactivity against 15mer peptides, but no specificity for the MART1 10mer. In contrast, T cells trained using a combination of conventional and preloaded DCs showed a mixed reactivity signature that included reactivity against TAA and MART1 15mers as well as the validated biologically relevant MART1 10mer. MART1 10mer specificity was confirmed by peptide-MHC tetramer staining (data not shown). The diversity of reactivity observed in the T cell product trained by a combination of conventional and preloaded DCs indicates that diversity of TAA-presenting DCs can support favorable diversity in the TAA-reactive T cells that DCs can stimulate for expansion.

### Example 4: Preloading Enables Isolation of MTC Reactive to 8-11mer from Commercial 15mer Libraries

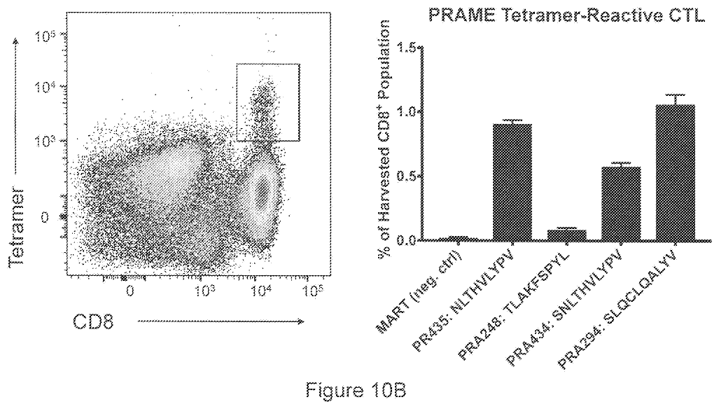

To identify MTCs that are reactive to smaller peptides in the harvested product, a combination method was run using a pool of 5 diversified 15mer peptide libraries comprising libraries of SSX2, NY-ESO1, PRAME, Survivin, and WT1. The pooled libraries consisted of 356 total peptides including 125 PRAME-derived peptides. Briefly, in the preloading method (FIG. 10A) these peptides were added to enriched monocytes in the presence of IL-4 and GM-CSF. After one day of culture to convert the monocytes to iDCs, the cells were incubated with TNF□, IL-6, IL-1□, and PGE2 for two additional days to convert the iDCs to mature DCs expressing 6-15mer peptides. In contrast, the conventional method first converted the monocytes to iDCs and then mDCs before loading with the 15mer peptide library pool. The preloaded and conventional DCs were mixed 1:1 and used to activate and expand a pool of T cells in a co-culture lasting 14 days with 10:1 T cell:total DC stimulations on days 0 and 7.

To identify MTC reactive to smaller processed peptides in the product, the harvested MTC were incubated with commercially-sourced HLA-A*02:01, fluorophore-conjugated tetramers loaded with selected PRAME-derived 9mer and 10mer peptides. MTC bearing TCR reactive to the 9mer and 10mer-loaded tetramers are identified through flow cytometry based on tetramer staining (FIG. 10B). The MART_{126-35(27L)} tetramer is used as a negative control for non-specific binding to tetramer. The study shows that clones that are reactive to immunologically significant 9 and 10mer can be isolated from off-the-shelf, highly diversified pools of peptides.

### Example 5: Preloaded and Conventional/Preloaded Combination DCs Stimulate and Expand T Cells that Recognize Antigen-Expressing Cancer Cells

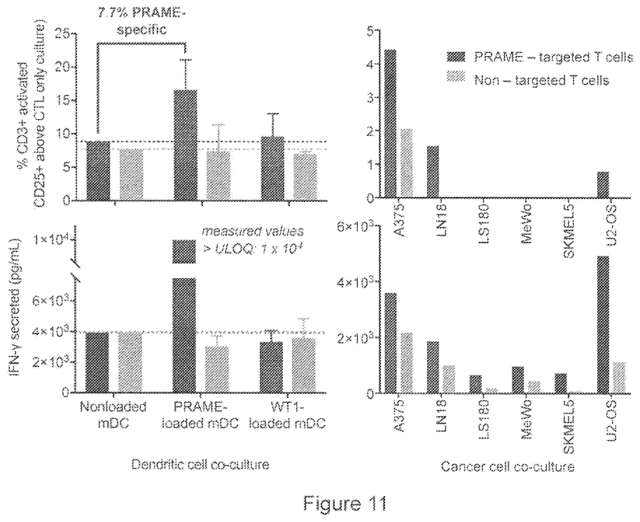

To assess the ability of TAA 15mer preloaded DCs to stimulate and expand TAA-reactive T cells that recognize TAA-expressing cancer cells, the preloading method was run using a library of 15mer peptides spanning PRAME. To provide a negative control for reactivity, unloaded DCs were used to stimulate the same starting T cell pool in a parallel process (e.g., an additional process was run in parallel, but no PRAME TAA peptide was added to the DCs). When evaluated for PRAME reactivity via co-culture with TAA loaded or unloaded DCs, 7.7% of PRAME-targeted T cells were specifically activated, leading to an increase in IFN-γ secretion above the ULOQ (1×10⁴ pg/mL) as measured by an IFN-γ ELISA (FIG. 11, left panels). In contrast, PRAME-targeted T cells showed no activation response to mDCs preloaded with the irrelevant TAA WT1, and T cells trained on unloaded DCs showed no antigen-specific activation or IFN-γ secretion. When cultured for 24 hours with partially HLA-matched PRAME+ cancer cell lines, PRAME-targeted T cells showed activation above baseline in response to 3/6 cancer cell lines (A375, LN18, and U2-OS). Although PRAME-targeted T cells showed variable IFN-γ secretion in cancer cell co-cultures, T cell activation was associated with increased IFN-γ secretion (FIG. 11, right panels). Non-targeted T cells only showed activation above baseline in response to A375 cells (potentially due to allogeneic interaction), and consistently secreted less IFN-γ than PRAME-targeted T cells.

### EQUIVALENTS

The present disclosure provides among other things novel methods and systems for preparing antigen-specific T lymphocytes. While specific embodiments of the subject disclosure have been discussed, the above specification is illustrative and not restrictive. Many variations of the disclosure will become apparent to those skilled in the art upon review of this specification. The full scope of the disclosure should be determined by reference to the claims, along with their full scope of equivalents, and the specification, along with such variations.

### INCORPORATION BY REFERENCE

Reference is made to International Patent Application Publication Nos. WO/2017/218533, WO/2019/050977, WO/2019/050978, WO2019/010224, WO/2019/010219, and WO/2019/010222. All publications, patents, published patent applications, and sequence database entries mentioned herein are hereby incorporated by reference in their entirety as if each individual publication or patent or sequence database entry is specifically and individually indicated to be incorporated by reference.

1. A method for preparing antigen-presenting cells (APCs), comprising:
(a) contacting a plurality of monocytes and/or immature dendritic cells with a library of peptides in a medium under suitable conditions for the monocytes and/or immature dendritic cells to internalize one or more of the library of peptides, wherein the library of peptides comprises peptide fragments of an antigen, and wherein the peptides are suitable for proteolytic processing by the monocytes and/or immature dendritic cells and subsequent loading onto at least one cell surface protein complex selected from a class I major histocompatibility complex (MHC I) and/or a class II major histocompatibility complex (MHC II); and
(b) culturing the monocytes and/or immature dendritic cells in the presence of one or more cytokines and/or growth factors under suitable conditions to induce differentiation of the monocytes and/or maturation of the immature dendritic cells into mature antigen-presenting dendritic cells, thereby to prepare APCs.

2. The method of claim 1, wherein the library comprises a plurality of peptides having a length of 5 or more, 8 or more, 10 or more, 15 or more, 20 or more, 5, 6, 7, 8, 9, 10, 11, 12, 13, 14, 15, 16, 17, 18, 19, 20, 10-15, 5-10, 5-15, 5-20, 8-10, 8-15, 8-20, 10-20, 15-20, 10-100, 10-150, 10-200, or longer than 200 amino acids.

3. The method of claim 1, wherein the library of peptides comprises fragments of more than one antigen.

4. The method of claim 1, wherein the library of peptides comprises fragments of 1, 2, 3, 4, 5, 6, 7, 8, 9, 10, 2-5, 2-10, 3-10, 4-10, 5-10, at least 1, at least 2, at least 3, at least 4, at least 5, or at least 6 antigens.

5. The method of claim 1, wherein step (a) comprises contacting the monocytes and/or immature dendritic cells with 2 or more libraries of peptides, wherein each library of peptides comprises fragments of a different antigen.

6. The method of claim 1, wherein step (a) comprises contacting the monocytes and/or immature dendritic cells with 1, 2, 3, 4, 5, 6, 7, 8, 9, 10, 2-5, 2-10, 3-10, 4-10, 5-10, at least 1, at least 2, at least 3, at least 4, at least 5, or at least 6 libraries of peptides.

7. The method of claim 1, wherein the antigen is a tumor-associated antigen.

8. The method of claim 1, wherein the antigen is a viral tumor-associated antigen.

9. The method of claim 4, wherein the antigens are tumor-associated antigens, viral tumor-associated antigens, or both.

10. The method of claim 6, wherein the libraries of peptides include a library of peptides derived from a tumor-associated antigen, a library of peptides derived from a viral tumor-associated antigen, or both.

11. The method of any of the preceding claims, further comprising:
(c) contacting the APCs with a library of peptides in a medium under suitable conditions for the APCs to load the peptides onto at least one cell surface protein complex selected from an MHC I and an MHC II, wherein the library of peptides comprises peptide fragments of an antigen, and
(d) optionally repeating step (c) one or more times.

12. The method of claim 11, wherein the library of peptides used in step (c) comprises a plurality of peptides having a length of 5 or more, 8 or more, 10 or more, 15 or more, 20 or more, 5, 6, 7, 8, 9, 10, 11, 12, 13, 14, 15, 16, 17, 18, 19, 20, 23, 25, 10-15, 5-10, 5-15, 5-20, 5-25, 8-10, 8-15, 8-25, 10-25, 15-25, 10-100, 10-150, 10-200, or longer than 200 amino acids.

13. The method of claim 11, wherein the library of peptides used in step (c) comprises peptide fragments of more than one antigen.

14. The method of claim 11, wherein the library of peptides used in step (c) comprises fragments of 1, 2, 3, 4, 5, 6, 7, 8, 9, 10, 2-5, 2-10, 3-10, 4-10, 5-10, at least 1, at least 2, at least 3, at least 4, at least 5, or at least 6 antigens.

15. The method of claim 11, wherein step (c) comprises contacting the APCs with 2 or more libraries of peptides, wherein each library of peptides comprises fragments of a different antigen.

16. The method of claim 15, wherein step (c) comprises contacting the APCs with 1, 2, 3, 4, 5, 6, 7, 8, 9, 10, 2-5, 2-10, 3-10, 4-10, 5-10, at least 1, at least 2, at least 3, at least 4, at least 5, or at least 6 libraries of peptides.

17. The method according to claim 16, wherein at least one of the library or libraries of peptides of step (c) is the same as at least one of the library or libraries of peptides of step (a).

18. The method according to claim 16, wherein at least one of the library or libraries of peptides of step (c) is different than at least one of the library or libraries of peptides of step (a).

19. The method of claim 11, wherein the library of peptides of step (c) comprises peptide fragments of a tumor-associated antigen, a viral tumor-associated antigen, or both.

20. The method of claim 16, wherein the libraries of peptides of step (c) include a library of peptides comprising fragments of a tumor-associated antigen, a library of peptides comprising fragments of a viral tumor-associated antigen, or both.

21. The method of claim 11, wherein step (a) comprises contacting the monocytes and/or immature dendritic cells with a library of peptides in a medium at a first concentration, and wherein step (c) comprises contacting the APCs with a library of peptides in a medium at a second concentration, wherein the first concentration is the same as, more than, or less than the second concentration.

22. The method according to claim 1, wherein the peptides comprise fragments of one or more tumor-associated antigens selected from the group consisting of PRAME, SSX2, NY-ESO-1, Survivin, WT-1 and MART.

23. The method according to any one of claims 1-10, further comprising contacting the APCs with a plurality of T cells under conditions suitable for antigen-priming and/or antigen-specific activation of the T cells, thereby to produce a population of T cells comprising primed and/or activated T cells specific for the antigen presented by the APCs.

24. The method of claim 23, wherein the APCs are contacted with the plurality of T cells in the presence of the at least one of the libraries of peptides of step (a) and/or step (c).

25. The method of claim 23, further comprising culturing the population of T cells in the presence of one or more cytokines and/or growth factors under conditions suitable to induce proliferation of the T cells.

26. The method of claim 25, wherein the population of T cells is cultured in the presence of at least one of the libraries of peptides of step (a) and/or step (c).

27. The method of claim 25, further comprising culturing the population of T cells in the presence of the APCs suitable for antigen-priming and/or antigen-specific activation of the T cells.

28. The method of claim 27, wherein the step of contacting the APCs with the primed and/or activated T cells is repeated one or more times.

29. The method of claim 25, wherein the primed and/or activated T cells are co-cultured with the APCs.

30. A method for preparing antigen-specific T cells, comprising:
(a) contacting a plurality of monocytes and/or immature dendritic cells with a library of peptides in a medium under suitable conditions for the monocytes and/or immature dendritic cells to internalize one or more of the peptides, wherein the library of peptides comprises peptide fragments of an antigen, and wherein the peptides are suitable for proteolytic processing by the monocytes and/or immature dendritic cells and subsequent loading onto at least one cell surface protein complex selected from a class I major histocompatibility complex (MHC I) and/or a class II major histocompatibility complex (MHC II);
(b) culturing the monocytes and/or immature dendritic cells in the presence of one or more cytokines and/or growth factors under suitable conditions to induce differentiation of the monocytes and/or maturation of the immature dendritic cells into mature antigen-presenting dendritic cells, thereby to prepare APCs;
(c) contacting a plurality of T cells with the APCs under conditions suitable for antigen-priming and/or antigen-specific activation of the T cells, thereby to prepare a population of T cells comprising primed and/or activated T cells specific for the antigen presented by the APCs;
(d) contacting the population of T cells prepared in step (c) with APCs under conditions to stimulate the primed and/or activated T cells; and
(e) optionally, repeating step (d) one or more times.

31. The method of claim 30, wherein step (c) comprises culturing the plurality of T cells with the APCs in the presence of a library of peptides.

32. The method of claim 31, wherein the library of peptides of step (c) is the same as the library of peptides of step (a).

33. The method of claim 31, wherein the library of peptides of step (c) is different than the library of peptides of step (a).

34. The method of claim 30, wherein step (d) comprises culturing the population of T cells with the APCs in the presence of a library of peptides.

35. The method of claim 34, wherein the library of peptides of step (d) is the same as the library of peptides of step (a).

36. The method of claim 34, wherein the library of peptides of step (d) is different than the library of peptides used in step (a).

37. A composition comprising a population of APCs prepared according to the method of any one of claims 1-22, wherein the APCs comprise a plurality of MHC I and/or MHC II complexes loaded with processed peptides, wherein preferably the processed peptides have been shortened in vivo by, e.g., iDCs before presentation on the MHC I and/or MHC II complexes.

38. The composition of claim 37, wherein the processed peptides are shorter in length than the peptides in the library of peptides, preferably less than 8, less than 10, less than 12, less than 15, between 5 and 15, between 8 and 10, between 8 and 12, between 8 and 14, or between 8 and 15 amino acids in length.

39. The composition of claim 37, further comprising a plurality of mDCs loaded with peptides from the library of peptides (e.g., TAAs).

40. A composition comprising a population of APCs prepared according to the method of any one of claims 1-22, wherein the population of APCs comprise a plurality of MHC I and/or MHC II complexes loaded with processed peptides, and a plurality of MHC I and/or MHC II complexes loaded with peptides from the library of peptides, wherein preferably the composition further comprises a plurality of mDCs loaded with TAAs.

41. A composition comprising a population of primed and/or activated T cells prepared according to the method of any one of claims 23-36, wherein the primed and/or activated T-cells comprise a plurality of MHC I and/or MHC II complexes loaded with processed peptides.

42. A composition comprising a population of primed and/or activated T cells prepared according to the method of any one of claims 23-36, wherein the primed and/or activated T-cells comprise a plurality of MHC I and/or MHC II complexes loaded with processed peptides, and a plurality of MHC I and/or MHC II complexes loaded with peptides from the library of peptides.

43. The composition of claim 41 or 42, wherein the population of primed and/or activated T cells comprise CD4+ T cells, CD8+ T cells, and/or cytotoxic T lymphocytes (CTLs).

44. A therapeutic composition comprising the composition of claim 41 or 42, and a pharmaceutically acceptable carrier or excipient.

45. A method of treating cancer comprising administering the therapeutic composition of claim 44 to a patient in need thereof.

46. A composition comprising:
a first population of APCs presenting a first plurality of peptides having at least 2 different lengths, wherein preferably the first plurality of peptides has been shortened in vivo by, e.g., iDCs before presentation on the first population of APCs; and
a second population of APCs presenting a second plurality of peptides having a uniform length.

47. The composition of claim 47, wherein the first population of APCs and the second population of APCs are present at a ratio of about 1:1.

48. A composition comprising an expanded population of monocyte-derived APCs, wherein each APC comprises at least one MHC complex loaded with a peptide from the same antigen, and wherein as a population, the APCs comprise a plurality of MHC complexes loaded with a plurality of peptides from said antigen, the plurality of peptides being of different lengths.

49. The composition of claim 48, wherein each of the plurality of peptides has a length of 5 or more, 6 or more, 7 or more, 8 or more, 10 or more, 15 or more, 20 or more, between 5 and 10, between 5 and 15, between 5 and 20, between 6 and 10, or between 6 and 20 amino acids.

50. The composition of claim 48 or 49, wherein as a population, the APC further comprise a second plurality of MHC complexes loaded with a plurality of peptides from a second antigen, the second plurality of peptides having the same length.

51. The composition of claim 50, wherein the first antigen and the second antigen are the same antigen.
